(19) United States
(12) Patent Application Publication
(10) Pub. No.: US 2017/0071991 A1
(43) Pub. Date: Mar. 16, 2017

(54) Oncolytic Virus and Aurora Kinase Inhibitor for the Treatment of Cancer

(71) Applicant: VIRTTU BIOLOGICS LIMITED, Glasgow (GB)

(72) Inventor: Joe Conner, Glasgow (GB)

(21) Appl. No.: 15/310,556

(22) PCT Filed: May 7, 2015
(86) PCT No.: PCT/GB2015/051347

§ 371 Date: Nov. 11, 2016

(30) Foreign Application Priority Data
May 12, 2014 (GB) 1408297.8

(51) Int. Cl. A61K 35/763; A61K 31/55; C12N 7/00

(52) U.S. Cl. CPC A61K 35/763; C12N 7/00; A61K 31/55; C12N 2710/16632

(57) ABSTRACT

The use of an oncolytic virus and an aurora kinase inhibitor in the treatment of cancer is disclosed.

### FIELD OF THE INVENTION

The present invention relates to the use of an oncolytic virus and one or both of an aurora kinase inhibitor or a histone deacetylase (HDAC) inhibitor in the treatment of cancer.

### BACKGROUND TO THE INVENTION

Oncolytic virotherapy concerns the use of lytic viruses which selectively infect and kill cancer cells. Some oncolytic viruses are promising therapies as they display exquisite selection for replication in cancer cells and their self-limiting propagation within tumors results in fewer toxic side effects. Several oncolytic viruses have shown great promise in the clinic (Bell, J., Oncolytic Viruses: An Approved Product on the Horizon? Mol Ther. 2010; 18(2): 233-234).

The Aurora kinases are serine/threonine kinases that functions as regulators of multiple facets of mitosis and cell division, including centrosome, duplication, mitotic spindle formation, chromosome alignment upon the spindle, mitotic checkpoint activation, and cytokinesis. There are three related mammalian Aurora kinases known as Aurora-A, Aurora-B, and Aurora-C. These kinases are overexpressed in a number of human cancers (Carvajal et al., Aurora Kinases: New Targets for Cancer Therapy. Clin Cancer Res 2006; 12 (23) Dec. 1, 2006).

Aurora-A localizes to the centrosome from centrosome duplication through mitotic exit and primarily functions in centrosome regulation and mitotic spindle formation. Dysregulation of Aurora has been linked to tumorigenesis. Aurora-A is located on chromosome 20q13.2, a region commonly amplified in malignancies, such as melanoma and cancers of the breast, colon, pancreas, ovaries, bladder, liver, and stomach. Interest in Aurora has intensified since the discovery that transfection of rodent Rat1 and NIH3T3 fibroblast cell lines with Aurora-A is sufficient to induce colony formation in culture and tumors in nude mice, thus establishing Aurora-A as a bone fide oncogene (Bischoff J R, Anderson L, ZhuY, et al. A homologue of Drosophila aurora kinase is oncogenic and amplified in human colorectal cancers. EMBO J 1998; 17:3052-65; Zhou H, Kuang J, Zhong L, et al. Tumour amplified kinase STK15/BTAK induces centrosome amplification, aneuploidy and transformation Nat Genet 1998; 20:189-93). The amino acid sequence of human Aurora kinase A can be found in Genbank under accession no. NP 003591.2 (GI:38327562).

Aurora-A is ubiquitously expressed and regulates cell cycle events occurring from late S phase through M phase, including centrosome maturation (Berdnik D, Knoblich J A. Drosophila Aurora-A is required for centrosome maturation and actin dependent asymmetric protein localization during mitosis. Curr Biol 2002; 12:640-7.), mitotic entry (Hirota T, Kunitoku N, Sasayama T, et al. Aurora-A and an interacting activator, the LIM protein Ajuba, are required for mitotic commitment in human cells. Cell 2003; 114:585-98; Dutertre S, Cazales M, Quaranta M, et al. Phosphorylation of CDC25B by Aurora-A at the centrosome contributes to the G2-M transition. J Cell Sci 2004; 117:2523-31.), centrosome separation (Marumoto T, Honda S, Hara T, et al. Aurora-A kinase maintains the fidelity of early and late mitotic events in HeLa cells. J Biol Chem 2003; 278:51786-95.), bipolar-spindle assembly (Kufer T A, Sillje H H, Korner R, Gruss O J, Meraldi P, Nigg E A. Human TPX2 is required for targeting Aurora-A kinase to the spindle. J Cell Biol 2002; 158:617-23; Eyers P A, Erikson E, Chen L G, Mailer J L. A novel mechanism for activation of the protein kinase Aurora A. Curr Biol 2003; 13:691-7.), chromosome alignment on the metaphase plate (Marumoto et al. supra; Kunitoku N, Sasayama T, Marumoto T, et al. CENP-A phosphorylation by Aurora-A in prophase is required for enrichment of Aurora-B at inner centromeres and for kinetochore function. Dev Cell 2003; 5:853-64.), cytokinesis (Marumoto et al. supra), and mitotic exit.

The role of Aurora-A in normal cellular physiology and tumorigenesis is further discussed in Marumoto T, Zhang D, Saya H. Aurora-A: a guardian of poles. Nat Rev Cancer 2005; 5:42-50.

Aurora-A overexpression is a necessary feature of Aurora-A induced tumorigenesis; however, both abnormal cellular localization and timing of Aurora-A expression are also implicated (Carvajal et al., Aurora Kinases: New Targets for Cancer Therapy. Clin Cancer Res 2006; 12 (23) Dec. 1, 2006).

Aurora-B is a subunit of the chromosomal passenger protein complex and functions to ensure accurate chromosome segregation and cytokinesis. Aurora-B undergoes dynamic localization during mitosis, localizing first to the inner centromeric region from prophase through metaphase and then to the spindle midzone and midbody from anaphase through cytokinesis. Aurora-B is located on chromosome 17p13.1, a region not typically amplified in human malignancies. Despite lack of amplification at the gene level, mRNA and protein levels of Aurora-B are frequently increased in tumors, such as colorectal cancer (Tatsuka M, Katayama H, Ota T, et al. Multinuclearity and increased ploidy caused by overexpression of the aurora- and Ipl1-like midbody-associated protein mitotic kinase in human cancer cells. Cancer Res 1998; 58:4811-6.). Exogenous overexpression of Aurora-B in Chinese hamster embryo cells results in subsequent chromosome separation defects during mitosis and increased invasiveness in vivo, suggesting a role for Aurora-B in tumorigenesis (Ota T, Suto S, Katayama H, et al. Increased mitotic phosphorylation of histone H3 attributable to AIM-1/Aurora-B overexpression contributes to chromosome number instability. Cancer Res 2002; 62: 5168-77.).

The amino acid sequence of human Aurora kinase B can be found in Genbank under accession no. NP_001243763.1 (GI:378786658).

Without Aurora-B activity, the mitotic checkpoint is compromised, resulting in increased numbers of aneuploid cells, genetic instability, and tumorigenesis (Weaver B A, Cleveland D W. Decoding the links between mitosis, cancer, and chemotherapy: the mitotic checkpoint, adaptation, and cell death. Cancer Cell 2005; 8:7-12).

Aurora-C is a chromosomal passenger protein and colocalizes with Aurora-B. Aurora-C is specifically expressed in the testis where it functions in spermatogenesis and regulation of cilia and flagella (Carvajal et al., Aurora Kinases: New Targets for Cancer Therapy. Clin Cancer Res 2006; 12 (23) Dec. 1, 2006).

Histone deacetylases (HDACs) regulate the expression and activity of numerous proteins that are involved in both cancer initiation and progression (Glozak, M A and Seto, E (2007). Histone deacetylases and cancer. Oncogene 26:5420-5432.). Several HDAC inhibitors have been shown to arrest the growth and/or induce apoptosis of cancer cells (Fouladi, M (2006). Histone deacetylase inhibitors in cancer therapy. Cancer Invest 24: 521-527; Marks, P, Rifkind, R A, Richon, V M, Breslow, R, Miller, T and Kelly, W K (2001); Histone deacetylases and cancer: causes and therapies. Nat Rev Cancer 1: 194-202).

Histone deacetylase (HDAC) inhibitors are a well characterised group of compounds. Indeed facilities such as HDAC Inhibitors Base (www.hdacis.com) provide extensive information on known HDAC inhibitors. HDAC inhibitors have been proposed for use in the treatment of a variety of cancers for several years (Vigushin et al., Anticancer Drugs 2002 January; 13(1):1-13).

The interaction of some oncolytic viruses with some HDAC inhibitors has been investigated by some research groups (Otsuki et al., Molecular Therapy Vol. 16, No. 9 1546-1555 September 2008; MacTavish et al., (2010) Enhancement of Vaccinia Virus Based Oncolysis with Histone Deacetylase Inhibitors. PLoS ONE 5(12); Ta-Chiang et al., Molecular Therapy Vol. 16 No. 6, 1041-1047, June 2008; WO2009/067808A1).

### SUMMARY OF THE INVENTION

The present invention concerns the use of an oncolytic virus to treat cancer, wherein the subject receives the oncolytic virus and a chemotherapeutic agent as part of the programme of treatment. The chemotherapeutic agent is preferably an epigenetic agent and more preferably is an aurora kinase inhibitor and/or a histone deacetylase (HDAC) inhibitor.

The oncolytic virus and chemotherapeutic agent are administered as part of a method of treating cancer in the subject. They may be administered simultaneously, e.g. as a combined preparation or as separate preparations one administered immediately after the other. Alternatively, they may be administered separately and sequentially, where one agent is administered and then the other administered later after a predetermined time interval.

In one aspect of the present invention an oncolytic virus is provided for use in a method of treating cancer, the method comprising simultaneous or sequential administration of an oncolytic virus and an aurora kinase inhibitor or a histone deacetylase (HDAC) inhibitor.

In another aspect of the present invention the use of an oncolytic virus in the manufacture of a medicament for use in a method of treatment of cancer is provided, wherein the method of treatment comprises administering an aurora kinase inhibitor or a histone deacetylase (HDAC) inhibitor to the patient in need of treatment.

In another aspect of the present invention a method of treating cancer is provided, the method comprising administration of an oncolytic virus and an aurora kinase inhibitor or a histone deacetylase (HDAC) inhibitor to a patient in need of treatment, thereby treating the cancer.

In another aspect of the present invention an oncolytic virus is provided for use in a method of treating cancer, wherein the method of treatment comprises administering an aurora kinase inhibitor or a histone deacetylase (HDAC) inhibitor to the patient in need of treatment.

In another aspect of the present invention an aurora kinase inhibitor or a histone deacetylase (HDAC) inhibitor is provided for use in a method of treating cancer, wherein the method of treatment comprises administering an oncolytic virus to the patient in need of treatment.

In another aspect of the present invention the use of an oncolytic virus in the manufacture of a medicament for use in a method of treatment of cancer is provided, wherein the method of treatment comprises administering an aurora kinase inhibitor or a histone deacetylase (HDAC) inhibitor to the patient in need of treatment.

In another aspect of the present invention the use of an aurora kinase inhibitor or a histone deacetylase (HDAC) inhibitor in the manufacture of a medicament for use in a method of treatment of cancer is provided, wherein the method of treatment comprises administering an oncolytic virus to the patient in need of treatment.

In a further aspect of the present invention a pharmaceutical composition or medicament is provided comprising an oncolytic virus and an aurora kinase inhibitor or a histone deacetylase (HDAC) inhibitor.

In some embodiments the oncolytic virus is an oncolytic herpes simplex virus. In some embodiments all copies of the ICP34.5 gene in the genome of the oncolytic herpes simplex virus are modified such that the ICP34.5 gene is incapable of expressing a functional ICP34.5 gene product. As such the oncolytic herpes simplex virus may be an ICP34.5 null mutant.

In some embodiments one or both of the ICP34.5 genes in the genome of the oncolytic herpes simplex virus are modified such that the ICP34.5 gene is incapable of expressing a functional ICP34.5 gene product.

In some embodiments the oncolytic herpes simplex virus is a mutant of HSV-1 strain 17. In preferred embodiments the oncolytic herpes simplex virus is HSV1716 (ECACC Accession No. V92012803). In some embodiments the herpes simplex virus is a mutant of HSV-1 strain 17 mutant 1716.

In other embodiments the oncolytic virus is selected from one of: an oncolytic reovirus, an oncolytic vaccinia virus, an oncolytic adenovirus, an oncolytic Coxsackie virus, an oncolytic Newcastle Disease Virus, an oncolytic parvovirus, an oncolytic poxvirus, an oncolytic paramyxovirus.

In another aspect of the present invention a kit comprising a predetermined amount of oncolytic virus and a predetermined amount of chemotherapeutic agent is provided, wherein the chemotherapeutic agent is an aurora kinase inhibitor or a histone deacetylase (HDAC) inhibitor. The kit may be provided together with instructions for the administration of the oncolytic virus, aurora kinase inhibitor and/or histone deacetylase (HDAC) inhibitor sequentially or simultaneously in order to provide a treatment for cancer.

In another aspect of the present invention products containing therapeutically effective amounts of:
(i) an oncolytic virus, preferably HSV1716, and
(ii) an aurora kinase inhibitor or a histone deacetylase (HDAC) inhibitor
   for simultaneous or sequential use in a method of medical treatment, preferably treatment of cancer, are provided. The products may be pharmaceutically acceptable formulations and may optionally be formulated as a combined preparation for coadministration.

### DESCRIPTION OF PREFERRED EMBODIMENTS

Oncolytic Viruses

The oncolytic virus may be any oncolytic virus. Preferably it is a replication-competent virus, being replication-competent at least in the target tumor cells. In some embodiments the oncolytic virus is selected from one of an oncolytic herpes simplex virus, an oncolytic reovirus, an oncolytic vaccinia virus, an oncolytic adenovirus, an oncolytic Newcastle Disease Virus, an oncolytic Coxsackie virus, an oncolytic measles virus. An oncolytic virus is a virus that will lyse cancer cells (oncolysis), preferably in a selective manner. Viruses that selectively replicate in dividing cells over non-dividing cells are often oncolytic. Oncolytic viruses are well known in the art and are reviewed in Molecular Therapy Vol. 18 No. 2 Feb. 2010 pg 233-234.

In some embodiments the oncolytic virus is a herpes simplex virus. The herpes simplex virus (HSV) genome comprises two covalently linked segments, designated long (L) and short (S). Each segment contains a unique sequence flanked by a pair of inverted terminal repeat sequences. The long repeat (RL or R_{L}) and the short repeat (RS or R_{S}) are distinct.

The HSV ICP34.5 (also called γ34.5) gene, which has been extensively studied, has been sequenced in HSV-1 strains F and syn17+ and in HSV-2 strain HG52. One copy of the ICP34.5 gene is located within each of the RL repeat regions. Mutants inactivating one or both copies of the ICP34.5 gene are known to lack neurovirulence, i.e. be avirulent/non-neurovirulent (non-neurovirulence is defined by the ability to introduce a high titre of virus (approx 10⁶ plaque forming units (pfu)) to an animal or patient without causing a lethal encephalitis such that the LD₅₀ in animals, e.g. mice, or human patients is in the approximate range of ≧10⁶ pfu), and be oncolytic.

Oncolytic HSV that may be used in the present invention include HSV in which one or both of the γ34.5 (also called ICP34.5) genes are modified (e.g. by mutation which may be a deletion, insertion, addition or substitution) such that the respective gene is incapable of expressing, e.g. encoding, a functional ICP34.5 protein. Preferably, in HSV according to the invention both copies of the γ34.5 gene are modified such that the modified HSV is not capable of expressing, e.g. producing, a functional ICP34.5 protein.

In some embodiments the oncolytic herpes simplex virus may be an ICP34.5 null mutant where all copies of the ICP34.5 gene present in the herpes simplex virus genome (two copies are normally present) are disrupted such that the herpes simplex virus is incapable of producing a functional ICP34.5 gene product. In other embodiments the oncolytic herpes simplex virus may lack at least one expressible ICP34.5 gene. In some embodiments the herpes simplex virus may lack only one expressible ICP34.5 gene. In other embodiments the herpes simplex virus may lack both expressible ICP34.5 genes. In still other embodiments each ICP34.5 gene present in the herpes simplex virus may not be expressible. Lack of an expressible ICP34.5 gene means, for example, that expression of the ICP34.5 gene does not result in a functional ICP34.5 gene product.

Oncolytic herpes simplex virus may be derived from any HSV including any laboratory strain or clinical isolate (non-laboratory strain) of HSV. In some preferred embodiments the HSV is a mutant of HSV-1 or HSV-2. Alternatively the HSV may be an intertypic recombinant of HSV-1 and HSV-2. The mutant may be of one of laboratory strains HSV-1 strain 17, HSV-1 strain F or HSV-2 strain HG52. The mutant may be of the non-laboratory strain JS-1. Preferably the mutant is a mutant of HSV-1 strain 17. The herpes simplex virus may be one of HSV-1 strain 17 mutant 1716, HSV-1 strain F mutant R3616, HSV-1 strain F mutant G207, HSV-1 mutant NV1020, or a further mutant thereof in which the HSV genome contains additional mutations and/or one or more heterologous nucleotide sequences. Additional mutations may include disabling mutations, which may affect the virulence of the virus or its ability to replicate. For example, mutations may be made in any one or more of ICP6, ICPO, ICP4, ICP27. Preferably, a mutation in one of these genes (optionally in both copies of the gene where appropriate) leads to an inability (or reduction of the ability) of the HSV to express the corresponding functional polypeptide. By way of example, the additional mutation of the HSV genome may be accomplished by addition, deletion, insertion or substitution of nucleotides.

A number of oncolytic herpes simplex viruses are known in the art. Examples include HSV1716, R3616 (e.g. see Chou & Roizman, Proc. Natl. Acad. Sci. Vol. 89, pp. 3266-3270, April 1992), G207 (Toda et al, Human Gene Therapy 9:2177-2185, Oct. 10, 1995), NV1020 (Geevarghese et al, Human Gene Therapy 2010 September; 21(9):1119-28), RE6 (Thompson et al, Virology 131, 171-179 (1983)), and Oncovex™ (Simpson et al, Cancer Res 2006; 66:(9) 4835-4842 May 1, 2006; Liu et al, Gene Therapy (2003): 10, 292-303).

In some preferred embodiments the herpes simplex virus is HSV-1 strain 17 mutant 1716 (HSV1716). HSV 1716 is an oncolytic, non-neurovirulent HSV and is described in EP 0571410, WO 92/13943, Brown et al (Journal of General Virology (1994), 75, 2367-2377) and MacLean et al (Journal of General Virology (1991), 72, 631-639). HSV 1716 has been deposited on 28 Jan. 1992 at the European Collection of Animal Cell Cultures, Vaccine Research and Production Laboratories, Public Health Laboratory Services, Porton Down, Salisbury, Wiltshire, SP4 0JG, United Kingdom under accession number V92012803 in accordance with the provisions of the Budapest Treaty on the International Recognition of the Deposit of Microorganisms for the Purposes of Patent Procedure (herein referred to as the ‘Budapest Treaty’).

In some embodiments the herpes simplex virus is a mutant of HSV-1 strain 17 modified such that both ICP34.5 genes do not express a functional gene product, e.g. by mutation (e.g. insertion, deletion, addition, substitution) of the ICP34.5 gene, but otherwise resembling or substantially resembling the genome of the wild type parent virus HSV-1 strain 17+. That is, the virus may be a variant of HSV1716, having a genome mutated so as to inactivate both copies of the ICP34.5 gene of HSV-1 strain 17+ but not otherwise altered to insert or delete/modify other protein coding sequences.

Other types of oncolytic virus are also known in the art. These include oncolytic poxvirus (e.g. orthopoxviruses) such as vaccinia virus JX-954 and GLV-1h68 (Park, B H et al. (2008) Lancet Oncol 9:533-542; Kelly et al. Human Gene Therapy 19:744-782 (August 2008); Wennier et al. Expert Rev Mol Med. 13 e18 5 Dec. 2011) oncolytic reovirus such as oncolytic reovirus type 3 Dearing (Pandha, H S, et al. (2009) Clin Cancer Res 15:6158-6166; Vidal, L et al. (2008) Clin Cancer Res 14:7127-7137), oncolytic adenovirus such as Onyx-015 (Cohen and Rudin. Curr Opin Investig Drugs 2001 December; 2(12):1770-5), oncolytic paramyxovirus such as oncolytic measles virus MV-Edm (Nakamura, T, et al. (2005) Nat Biotechnol 23: 209-214; Wennier et al. Expert Rev Mol Med. 13 e18 5 Dec. 2011), oncolytic Coxsackie virus such as A13, A15, A18, A21 (Au et al, Virology Journal 2011, 8:22), oncolytic Newcastle Disease Virus (Mansour et al, J Virol 2011, June; 85(12):6015-23), and oncolytic parvoviruses such as H-1 PV and MVM (Wennier et al. Expert Rev Mol Med. 13 e18 5 Dec. 2011).

In some embodiments the genome of an oncolytic virus according to the present invention may be further modified to contain nucleic acid encoding at least one copy of a polypeptide that is heterologous to the virus (i.e. is not normally found in wild type virus) such that the polypeptide can be expressed from the nucleic acid. As such, the oncolytic virus may also be an expression vector from which the polypeptide may be expressed. Examples of such viruses are described in WO2005/049846 and WO2005/049845.

In order to effect expression of the polypeptide, nucleic acid encoding the polypeptide is preferably operably linked to a regulatory sequence, e.g. a promoter, capable of effecting transcription of the nucleic acid encoding the polypeptide. A regulatory sequence (e.g. promoter) that is operably linked to a nucleotide sequence may be located adjacent to that sequence or in close proximity such that the regulatory sequence can effect and/or control expression of a product of the nucleotide sequence. The encoded product of the nucleotide sequence may therefore be expressible from that regulatory sequence.

Oncolytic viruses may be formulated as medicaments and pharmaceutical compositions for clinical use and in such formulations may be combined with a pharmaceutically acceptable carrier, diluent or adjuvant. The composition may be formulated for topical, parenteral, systemic, intracavitary, intravenous, intra-arterial, intramuscular, intrathecal, intraocular, intratumoral, subcutaneous, oral or transdermal routes of administration which may include injection. Suitable formulations may comprise the virus in a sterile or isotonic medium. Medicaments and pharmaceutical compositions may be formulated in fluid (including gel) or solid (e.g. tablet) form. Fluid formulations may be formulated for administration by injection or via catheter to a selected region of the human or animal body.

Administration is preferably in a “therapeutically effective amount”, this being sufficient to show benefit to the individual. The actual amount administered, and rate and time-course of administration, will depend on the nature and severity of the disease being treated. Prescription of treatment, e.g. decisions on dosage etc, is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Remington's Pharmaceutical Sciences, 20th Edition, 2000, pub. Lippincott, Williams & Wilkins.

Targeting therapies may be used to deliver the oncolytic virus to certain types of cell, e.g. by the use of targeting systems such as antibody or cell specific ligands. Targeting may be desirable for a variety of reasons; for example if the virus is unacceptably toxic in high dose, or if it would otherwise require too high a dosage, or if it would not otherwise be able to enter the target cells.

HSV capable of targeting cells and tissues are described in (PCT/GB2003/000603; WO 03/068809), hereby incorporated in its entirety by reference.

An oncolytic virus may be administered alone or in combination with other treatments, either simultaneously or sequentially dependent upon the condition to be treated. Such other treatments may include chemotherapy (including either systemic treatment with a chemotherapeutic agent or targeted therapy using small molecule or biological molecule (e.g. antibody) based agents that target key pathways in tumor development, maintenance or progression) or radiotherapy provided to the subject as a standard of care for treatment of the cancer.

Chemotherapy

Chemotherapy refers to treatment of a tumor with a drug. For example, the drug may be a chemical entity, e.g. small molecule pharmaceutical, protein inhibitor (e.g. kinase inhibitor), or a biological agent, e.g. antibody, antibody fragment, nucleic acid or peptide aptamer, nucleic acid (e.g. DNA, RNA), peptide, polypeptide, or protein. The drug may be formulated as a pharmaceutical composition or medicament. The formulation may comprise one or more drugs (e.g. one or more active agents) together with one or more pharmaceutically acceptable diluents, excipients or carriers.

A treatment may involve administration of more than one drug. A drug may be administered alone or in combination with other treatments, either simultaneously or sequentially dependent upon the condition to be treated. For example, the chemotherapy may be a co-therapy involving administration of two drugs/agents, one or more of which may be intended to treat the tumor. In the present invention an oncolytic virus and chemotherapeutic may be administered simultaneously, separately, or sequentially which may allow the two agents to be present in the tumor requiring treatment at the same time and thereby provide a combined therapeutic effect, which may be additive or synergistic.

The chemotherapy may be administered by one or more routes of administration, e.g. parenteral, intra-arterial injection or infusion, intravenous injection or infusion, intraperitoneal, intratumoral or oral. Administration is preferably in a “therapeutically effective amount”, this being sufficient to show benefit to the individual. The actual amount administered, and rate and time-course of administration, will depend on the nature and severity of the disease being treated. Prescription of treatment, e.g. decisions on dosage etc, is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Remington's Pharmaceutical Sciences, 20th Edition, 2000, pub. Lippincott, Williams & Wilkins.

The chemotherapy may be administered according to a treatment regime. The treatment regime may be a pre-determined timetable, plan, scheme or schedule of chemotherapy administration which may be prepared by a physician or medical practitioner and may be tailored to suit the patient requiring treatment.

The treatment regime may indicate one or more of: the type of chemotherapy to administer to the patient; the dose of each drug; the time interval between administrations; the length of each treatment; the number and nature of any treatment holidays, if any etc. For a co-therapy a single treatment regime may be provided which indicates how each drug/agent is to be administered.

Aurora Kinase Inhibitors

Aurora kinase inhibitors are examples of chemotherapeutic agents that may be used together with an oncolytic virus to treat a cancer in accordance with the present invention.

An aurora kinase inhibitor is an agent capable of inhibiting the activity of an Aurora kinase, preferably a mammalian Aurora kinase, more preferably a human Aurora kinase. In some preferred embodiments the agent is capable of inhibiting the activity of human Aurora kinase A and/or B and/or C.

An aurora kinase inhibitor will typically be a chemical entity, e.g. small molecule pharmaceutical, antibiotic, or a biological agent, e.g. antibody, antibody fragment, nucleic acid or peptide aptamer, nucleic acid (e.g. DNA, RNA), peptide, polypeptide, or protein. In normal cells, Aurora-A inhibition results in delayed, but not blocked, mitotic entry (Hirota T, Kunitoku N, Sasayama T, et al. Aurora-A and an interacting activator, the LIM protein Ajuba, are required for mitotic commitment in human cells. Cell 2003; 114:585-98; Marumoto T, Honda S, Hara T, et al. Aurora-A kinase maintains the fidelity of early and late mitotic events in HeLa cells. J Biol Chem 2003; 278:51786-95.); centrosome separation defects resulting in unipolar mitotic spindles (Marumoto et al, supra; Glover D M, Leibowitz M H, McLean D A, Parry H. Mutations in aurora prevent centrosome separation leading to the formation of monopolar spindles. Cell 1995; 81:95-105.); and failure of cytokinesis (Marumoto et al, supra). Encouraging antitumor effects with Aurora-A inhibition have been shown in three human pancreatic cancer cell lines (Panc-1, MIA PaCa-2, and SU.86.86), with growth suppression in cell culture and near-total abrogation of tumorigenicity in mouse xenografts (Hata T, Furukawa T, Sunamura M, et al. RNA interference targeting aurora kinase A suppresses tumor growth and enhances the taxane chemosensitivity in human pancreatic cancer cells. Cancer Res 2005; 65:2899-905).

Aurora-B inhibition results in abnormal kinetochore-microtubule attachments, failure to achieve chromosomal biorientation, and failure of cytokinesis (Goto H, Yasui Y, Kawajiri A, et al. Aurora-B regulates the cleavage furrow-specific vimentin phosphorylation in the cytokinetic process. J Biol Chem 2003; 278:8526-30; Severson A F, Hamill D R, Carter J C, Schumacher J, Bowerman B. The aurora-related kinase AIR-2 recruits ZEN-4/CeMKLP1 to the mitotic spindle at metaphase and is required for cytokinesis. Curr Biol 2000; 10:1162-71). The mitotic checkpoint is compromised, allowing cells to progress through mitosis despite incorrect microtubule-kinetochore attachments (Hauf S, Cole R W, LaTerra S, et al. The small molecule Hesperadin reveals a role for Aurora B in correcting kinetochore-microtubule attachment and in maintaining the spindle assembly checkpoint. J Cell Biol 2003; 161:281-94; Kallio M J, McCleland M L, Stukenberg P T, Gorbsky G J. Inhibition of aurora B kinase blocks chromosome segregation, overrides the spindle checkpoint, and perturbs microtubule dynamics in mitosis. Curr Biol 2002; 12:900-5.). Although the initial recruitment of checkpoint proteins, such as BubR1 and Mad2, to kinetochores occurs normally during prophase, they subsequently dissociate as mitosis progresses in the absence of Aurora-B function. This dissociation weakens the checkpoint, allowing cells undergoing abnormal mitosis to progress from metaphase to anaphase. Recurrent cycles of aberrant mitosis without cytokinesis result in massive polyploidy and, ultimately, to apoptosis (Hauf et al, supra; Ditchfield et al, supra; Giet R, Glover D M. Drosophila aurora B kinase is required for histone H3 phosphorylation and condensing recruitment during chromosome condensation and to organize the central spindle during cytokinesis. J Cell Biol 2001; 152:669-82; Murata-Hori M, Wang Y L. The kinase activity of aurora B is required for kinetochore-microtubule interactions during mitosis. Curr Biol 2002; 12:894-9; Kallio et al, supra).

Inhibition of Aurora-A or Aurora-B activity in tumor cells results in impaired chromosome alignment, abrogation of the mitotic checkpoint, polyploidy, and subsequent cell death. These in vitro effects are greater in transformed cells than in either non-transformed or non-dividing cells (Ditchfield et al, supra). Thus, targeting Aurora may achieve in vivo selectivity for cancer.

Inhibition of Aurora kinase activity can be tested using routine procedures known to those of ordinary skill in the art, thus allowing one to confirm whether a given agent is an Aurora kinase inhibitor. Suitable methods include the use of in vitro kinase assays, such as those described in Harrington E A, Bebbington D, Moore J, et al. VX-680, a potent and selective small-molecule inhibitor of the Aurora kinases, suppresses tumor growth in vivo. Nat Med 2004; 10:262-7. Alternatively, a commercially available kit designed for screening of Aurora kinase inhibitors may be selected such as the CycLex® Aurora A Kinase Assay/Inhibitor Screening Kit (MBL corporation, CY-1165) or CycLex® Aurora Family Kinase Assay/Inhibitor Screening Kit (MBL corporation, CY-1174).

Many aurora kinase inhibitors are known, as discussed below.

MLN8237

MLN8237 (9-chloro-7-(2-fluoro-6-methoxyphenyl)-5H-pyrimido[5,4-d][2]benzazepin-2-yl)-amino]-2-methoxybenzoic acid) (Millenium Pharmaceuticals, Cambridge, Mass.) is an Aurora Kinase A inhibitor. MLN8237 is an orally active small-molecule inhibitor of Aurora A kinase. It is a selective Aurora A inhibitor having an IC50 value of 1 nM with a 200-fold selectivity for Aurora A over Aurora B in cell-based studies (Karthigeyan et al., Medicinal Research Reviews Volume 31, Issue 5, pages 757-793, September 2011).

The structure of MLN8237 is shown in FIG. 1.

Hesperadin

Hesperadin is an indolinone that inhibits immunoprecipitated Aurora-B with an inhibitory concentration 50% (IC50) of 250 nmol/L (Carvajal et al., Aurora Kinases: New Targets for Cancer Therapy. Clin Cancer Res 2006; 12 (23) Dec. 1, 2006). It induces aberrant microtubule kinetochore attachments, with a significant increase in the formation of syntelic attachments (Hauf S, Cole R W, LaTerra S, et al. The small molecule Hesperadin reveals a role for Aurora B in correcting kinetochore-microtubule attachment and in maintaining the spindle assembly checkpoint. J Cell Biol 2003; 161:281-94). Despite failing to achieve proper chromosome biorientation, treated cells evade the mitotic checkpoint and proceed from metaphase to anaphase (Hauf et al, supra; Harrington E A, Bebbington D, Moore J, et al. VX-680, a potent and selective small-molecule inhibitor of the Aurora kinases, suppresses tumor growth in vivo. Nat Med 2004; 10:262-7). These cells fail to undergo cytokinesis and tetraploidy results. Despite the increased polyploidy, no loss of cell viability is achieved.

The structure of Hesperadin is shown in FIG. 1.

ZM447439

ZM447439 is a quinazoline derivative developed by AstraZeneca that is an ATP competitor of Aurora. In vitro assays show inhibition of both Aurora-A and Aurora-B with an IC50 of f100 nmol/L (Carvajal et al., Aurora Kinases: New Targets for Cancer Therapy. Clin Cancer Res 2006; 12 (23) Dec. 1, 2006).

As with Hesperadin, ZM447439 induces incorrect microtubule kinetochore attachments, failure of chromosome biorientation, abrogation of the mitotic checkpoint, failure of cytokinesis, and the development of tetraploidy (Hauf S, Cole R W, LaTerra S, et al. The small molecule Hesperadin reveals a role for Aurora B in correcting kinetochore-microtubule attachment and in maintaining the spindle assembly checkpoint. J Cell Biol 2003; 161:281-94; Harrington E A, Bebbington D, Moore J, et al. VX-680, a potent and selective small-molecule inhibitor of the Aurora kinases, suppresses tumor growth in vivo Nat Med 2004; 10:262-7). Treated cells undergo apoptosis with the next cell cycle. Exposure to ZM447439 achieves both growth inhibition and apoptosis. Although ZM447439 inhibits both Aurora-A and Aurora-B in vitro, the phenotype observed in treated cells suggests a greater inhibition of Aurora-B.

The structure of ZM447439 is shown in FIG. 1.

MK0457 (also called VX-680)

MK0457 inhibits all three Aurora kinases. Each induces a similar phenotype in cell-based assays, characterized by inhibition of phosphorylation of histone H3 on Ser10, inhibition of cytokinesis, and the development of polyploidy (Hauf S, Cole R W, LaTerra S, et al. The small molecule Hesperadin reveals a role for Aurora B in correcting kinetochore-microtubule attachment and in maintaining the spindle assembly checkpoint. J Cell Biol 2003; 161:281-94; Ditchfield C, Johnson V L, Tighe A, et al. Aurora B couples chromosome alignment with anaphase by targeting BubR1, Mad2, and Cenp-E to kinetochores. J Cell Biol 2003; 161:267-80; Harrington E A, Bebbington D, Moore J, et al. VX-680, a potent and selective small-molecule inhibitor of the Aurora kinases, suppresses tumor growth in vivo Nat Med 2004; 10:262-7).

MK0457 is a 4,6 diaminopyrimidine that targets the ATP-binding site common to all Aurora kinases. It is a potent inhibitor of all three Aurora kinases, with inhibition constants (Ki) of 0.6, 18.0, and 4.6 nmol/L for Aurora-A, Aurora-B, and Aurora-C, respectively (Harrington et al, supra).

Treatment with MK0457 results in polyploidy and additionally inhibits the growth of several tumor types in cell culture, with the induction of apoptosis most prominent in leukemia, lymphoma, and colorectal cell lines. Studies of MK0457 in rodent xenograft models of leukemia, colon cancer, and pancreatic cancer also show impressive antitumor activity.

Treatment of human acute myelogenous leukemia (HL60) nude mice xenografts with MK0457 resulted in a 98% reduction in tumor volume when compared with controls (Harrington et al, supra). In a human colon cancer (HCT116) nude rat xenograft model, treatment with MK0457 resulted in tumor regression in four of the seven rats treated. In all treated xenografts, phosphorylation of histone H3 at Ser10 was inhibited, indicating effective Aurora-B inhibition.

The structure of MK0457 is shown in FIG. 1.

MLN8054

MLN8054 is an oral small-molecule inhibitor of Aurora with relative specificity for Aurora-A (Aurora-A IC50=0.034 Amol/L; Aurora-B IC50=5.7 Amol/L; [Hoar H M, Wysong D R, Ecsedy J A. MLN8054 selectively inhibits Aurora A overAurora B in cultured human tumor cells [abstract C40]. Proc AACR-NCIEORTC International Conference: Molecular Targets and Cancer Therapeutics 2005.]). MLN8054 is a selective Aurora-A inhibitor.

Treatment of cultured human tumor cells with low concentrations of MLN8054 (0.25-2 Amol/L) results in aberrant mitotic spindle formation consistent with Aurora-A inhibition. Treatment at higher concentrations (4 Amol/L) results in loss of phosphorylation of histone H3 on Ser10, consistent with Aurora-B inhibition. Growth inhibition was shown in HCT116 human colon cancer, PC4 prostate cancer, and Calu-6 human lung cancer xenograft models using various oral dosing schedules (Huck J, Zhang M, Burenkova O, Connolly K, Manfredi M, Meetze K. Preclinical antitumor activity with MLN8054, a small molecule Aurora A kinase inhibitor [abstract 4698]. Proc Am Assoc Cancer Res 2006; 47:1104.).

Compound 677

Compound 677 is a selective Aurora-B inhibitor developed by AstraZeneca. It shows potent single-agent anticancer activity in preclinical studies (Nair J S, Tse A, Keen N, Schwartz G K. A novel aurora B kinase inhibitor with potent anticancer activity either as a single agent or in combination with chemotherapy [abstract 9568]. Proc Am Soc Clin Oncol. 2004; 23:848.).

AZD1152

AZD1152 is a selective Aurora-B inhibitor developed by AstraZeneca. It is a highly soluble acetanilide-substituted pyrazole-aminoquinazolone pro-drug that is cleaved completely in human plasma to yield the active drug substance AZD1152 hydroxy-QPA. AZD1152 hydroxy-QPA inhibits Aurora-A, Aurora B-INCENP, and Aurora C-INCENP with respective inhibitory coefficients of 687, 3.7, and 17.0 nmol/L, indicating a 100-fold selectivity for Aurora-B over Aurora-A (Carvajal et al., Aurora Kinases: New Targets for Cancer Therapy. Clin Cancer Res 2006; 12 (23) Dec. 1, 2006).

Cell line studies reveal inhibition of histone H3 phosphorylation at Ser10 and progression with normal kinetics through an aberrant mitosis, resulting in polyploidy and cell death. Xenograft studies of AZD1152 show reduced phosphorylation of histone H3 on Ser10, increased polyploidy and enhanced apoptosis in athymic nude rodents bearing various human tumors, including colorectal cancer (SW620, HCT116, and Colo205) and lung cancer (A549 and Calu-6; [Wilkinson R W, Odedra R, Heaton S P, et al. AZD1152, highly potent Aurora kinase inhibitor, with selectivity for Aurora kinase B, induces pharmacodynamics effects and significant growth inhibition in human tumor xenograft models [abstract B214]. Proc AACR-NCI-EORTC International Conference: Molecular Targets and Cancer Therapeutics 2005(183).]).

Further Aurora kinase inhibitors include: PHA-680632 ((Nerviano Medical Sciences); Soncini C, Carpinelli P, Gianellini L, et al. PHA-680632, a novel Aurora kinase inhibitor with potent antitumoral activity. Clin Cancer Res 2006; 12:4080-9.), PHA-739358 (Nerviano Medical Sciences), R763 (Rigel; McLaughlin et al., J Cancer Research Clin Oncol. 2010 January; 136(1):99-113), SNS-314 (Sunesis; Arbitrario et al., Cancer Chemother Pharmacol. 2010 March; 65(4):707-17. Epub 2009 Aug. 1.), NCED#17 (NCE Discovery Ltd), AT9283 (Astex Therapeutics; Dawson et al., Br J Haematol. 2010 July; 150(1):46-57. Epub 2010 May 7.), MP-235 (Montigen Pharmaceuticals), MP-529 (Montigen Pharmaceuticals), MLN8054 (Millenium; (Karthigeyan et al., Medicinal Research Reviews Volume 31, Issue 5, pages 757-793, September 2011), PHA-739358 (Karthigeyan et al., Medicinal Research Reviews Volume 31, Issue 5, pages 757-793, September 2011), as well as those illustrated in FIG. 1 and those described in WO2007/113005 and WO2007/115805, including compound VII described in WO2007/113005 and compounds IV, VII, VIII and IX described in WO2007/115805 (and illustrated in FIG. 1).

Histone Deacetylase (HDAC) Inhibitors

Histone deacetylase (HDAC) inhibitors are examples of chemotherapeutic agents that may be used together with an oncolytic virus to treat a cancer in accordance with the present invention.

HDAC inhibitors are compounds that inhibit the enzymatic activity of histone deacetylase. An HDAC inhibitor will typically be a chemical entity, e.g. small molecule pharmaceutical, antibiotic, or a biological agent, e.g. antibody, antibody fragment, nucleic acid or peptide aptamer, nucleic acid (e.g. DNA, RNA), peptide, polypeptide, or protein.

HDAC inhibitors are capable of inducing growth arrest, differentiation and/or apoptosis of cancer cells ex vivo, as well as in vivo in tumor-bearing animal models. Several classes of HDAC inhibitors are undergoing clinical trials as anti-tumor agents.

HDAC inhibitor activity can be tested using routine procedures known to those of ordinary skill in the art, thus allowing one to confirm whether a given agent is an HDAC inhibitor. Suitable methods include the use of in vitro assays, such as those described in Hoffman et al (Nucleic Acids Research, 1999, Vol. 27, No. 9, 2057-2058). Alternatively, several commercially available kits designed for screening for HDAC inhibitors are available, such as the HDAC Assay Kits (cat no.s 56200 and 56210) supplied by Active Motif (Carlsbad, Calif., USA).

Many HDAC inhibitors are known, as discussed below.

Romidepsin

Romidepsin (trade name Istodax® (Celgene); codenamed FK228 and FR901228) approved as a treatment for cutaneous T-cell lymphoma (CTCL) and is undergoing Phase 2 clinical trials for treatment of Non-Hodgkin Lymphoma (NHL).

The structure of Romidepsin is shown in FIG. 2.

Vorinostat

Vorinostat (or suberoylanilide hydroxamic acid (SAHA)), is marketed under the name Zolinza® for the treatment of cutaneous T cell lymphoma (CTCL). Vorinostat is undergoing Phase 3 clinical trials for the treatment of mesothelioma and phase 2 trials for the treatment of MDS, NHL, brain cancer and NSCLC. Vorinostat in combination with bortezomib (Velcade®) is undergoing phase 2 and 3 clinical trials for multiple myeloma.

The structure of Vorinostat is shown in FIG. 2.

Panobinostat

Panobinostat (LBH589; Novartis) is a non-selective HDAC inhibitor undergoing Phase 3 clinical trials for the treatment of Hodgkin's lymphoma, chronic myelogenous leukemia (CML), acute myeloid leukemia (AML) and myelodysplastic syndrome (MDS).

Panobinostatin in combination with bortezomib and dexamethasone is undergoing phase 2/3 clinical trials for treatment of multiple myeloma.

The structure of Panobinostatin is shown in FIG. 2.

Belinostat

Belinostat (PXD101; Spectrum Pharmaceuticals, TopoTarget; Curagen) is undergoing Phase 2 clinical trials for treatment of AML, CTCL, MDS, NHL and ovarian cancer.

The structure of Belinostat is shown in FIG. 2.

Mocetinostat

Mocetinostat (MGCD0103; Mocetinostat dihydrobromide) is a benzamide histone deacetylase inhibitor undergoing clinical trials for treatment of AML, chronic lymphocytic leukemia (CLL), Hodgkin's lymphoma, NHL, pancreatic cancer and thymic carcinoma

The structure of Mocetinostat is shown in FIG. 2.

Entinostat

Entinostat (SNDX-275; Syndax Pharmaceuticals) is a benzamide histone deacetylase inhibitor undergoing clinical trials for treatment of Breast cancer, Hodgkin's lymphoma and NSCLC.

The structure of Entinostat is shown in FIG. 2.

PCI-24781

PCI-24781 (CRA-02478; Pharmacyclics) is undergoing clinical trials for treatment of haematologic cancer and sarcoma.

Examples of other HDACs include valproic acid, trichostatin A, Apicidin, LBH-589, CS-00028 (Chipscreen Biosciences), CHR-2504 (Chroma Therapeutics), FR-135313 (Gloucester Pharmaceuticals), JNJ-16241199 (Johnson & Johnson), MGCD0103 (Methylgene), LAQ-824 (Novartis), LBH-589 (Novartis), CC1994 (Pfizer), MS 275 (Schering), and pivaloyloxymethyl butyrate (Titan Pharmaceuticals).

A number of HDAC inhibitors are also described in AU 2001/18768 B2, AU 2002/327627 B2, U.S. Pat. No. 6,897,220, U.S. Pat. No. 0,039,850, U.S. Pat. No. 6,541,661, U.S. Pat. No. 7,288,567, U.S. Pat. No. 7,253,204, AU 2001/283925 B2, U.S. Pat. No. 7,282,608, U.S. Pat. No. 7,250,514, U.S. Pat. No. 7,169,801, U.S. Pat. No. 7,154,002, U.S. Pat. No. 6,495,719, U.S. Pat. No. 7,057,057, U.S. Pat. No. 7,214,831, U.S. Pat. No. 7,191,305, U.S. Pat. No. 7,126,001, U.S. Pat. No. 7,205,304, EP 12068086 B1, U.S. Pat. No. 6,511,990, U.S. Pat. No. 7,244,751, AU 2002/246053 B2, AU 25 2000/68416 B2, U.S. Pat. No. 7,091,229, U.S. Pat. No. 6,638,530, EP 1501508 B1, EP 1656348 B1, EP 1358168 B1, U.S. Pat. No. 7,067,551, AU 2001/282129 B2, U.S. Pat. No. 6,552,065, U.S. Pat. No. 683,384, EP 1301184 B1, EP 1318980 B1, U.S. Pat. No. 6,960,685, U.S. Pat. No. 6,888,027, EP 1335898 B1, U.S. Pat. No. 7,183,298, U.S. Pat. No. 7,135,493, U.S. Pat. No. 6,825,317, and U.S. Pat. No. 6,656,905.

Forms of Chemotherapeutic Agent

The active compound of a given chemotherapeutic agent may be provided in the form of a corresponding salt, solvate, or prodrug. In this specification reference to the chemotherapeutic agent includes reference to such forms.

Salts

It may be convenient or desirable to prepare, purify, and/or handle a corresponding salt of the active compound, for example, a pharmaceutically-acceptable salt. Examples of pharmaceutically acceptable salts are discussed in Berge et al., 1977, “Pharmaceutically Acceptable Salts,” J. Pharm. Sci., Vol. 66, pp. 1-19.

For example, if the compound is anionic, or has a functional group which may be anionic (e.g., —COOH may be —COO⁻), then a salt may be formed with a suitable cation. Examples of suitable inorganic cations include, but are not limited to, alkali metal ions such as Na⁺ and K⁺, alkaline earth cations such as Ca²⁺ and Mg²⁺, and other cations such as Al⁺³. Examples of suitable organic cations include, but are not limited to, ammonium ion (i.e., NH₄⁺) and substituted ammonium ions (e.g., NH₃R⁺, NH₂R₂⁺, NHR₃⁺, NR₄⁺). Examples of some suitable substituted ammonium ions are those derived from: ethylamine, diethylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. An example of a common quaternary ammonium ion is N(CH₃)₄⁺.

If the compound is cationic, or has a functional group which may be cationic (e.g., —NH₂ may be —NH₃⁺), then a salt may be formed with a suitable anion. Examples of suitable inorganic anions include, but are not limited to, those derived from the following inorganic acids: hydrochloric, hydrobromic, hydroiodic, sulfuric, sulfurous, nitric, nitrous, phosphoric, and phosphorous.

Examples of suitable organic anions include, but are not limited to, those derived from the following organic acids: 2-acetyoxybenzoic, acetic, ascorbic, aspartic, benzoic, camphorsulfonic, cinnamic, citric, edetic, ethanedisulfonic, ethanesulfonic, fumaric, glucheptonic, gluconic, glutamic, glycolic, hydroxymaleic, hydroxynaphthalene carboxylic, isethionic, lactic, lactobionic, lauric, maleic, malic, methanesulfonic, mucic, oleic, oxalic, palmitic, pamoic, pantothenic, phenylacetic, phenylsulfonic, propionic, pyruvic, salicylic, stearic, succinic, sulfanilic, tartaric, toluenesulfonic, and valeric. Examples of suitable polymeric organic anions include, but are not limited to, those derived from the following polymeric acids: tannic acid, carboxymethyl cellulose.

Unless otherwise specified, a reference to a particular compound also include salt forms thereof.

Solvates

It may be convenient or desirable to prepare, purify, and/or handle a corresponding solvate of the active compound. The term “solvate” is used herein in the conventional sense to refer to a complex of solute (e.g., active compound, salt of active compound) and solvent. If the solvent is water, the solvate may be conveniently referred to as a hydrate, for example, a mono-hydrate, a di-hydrate, a tri-hydrate, etc.

Unless otherwise specified, a reference to a particular compound also include solvate forms thereof.

Prodrugs

It may be convenient or desirable to prepare, purify, and/or handle the active compound in the form of a prodrug. The term “prodrug,” as used herein, pertains to a compound which, when metabolised (e.g., in vivo), yields the desired active compound. Typically, the prodrug is inactive, or less active than the active compound, but may provide advantageous handling, administration, or metabolic properties.

Unless otherwise specified, a reference to a particular compound also include prodrugs thereof.

For example, some prodrugs are esters of the active compound (e.g., a physiologically acceptable metabolically labile ester). During metabolism, the ester group (—C(═O)OR) is cleaved to yield the active drug. Such esters may be formed by esterification, for example, of any of the carboxylic acid groups (—C(═O)OH) in the parent compound, with, where appropriate, prior protection of any other reactive groups present in the parent compound, followed by deprotection if required.

Examples of such metabolically labile esters include those of the formula —C(═O)OR wherein R is: C₁₋₇alkyl (e.g., -Me, -Et, -nPr, -iPr, -nBu, -sBu, -iBu, -tBu); C₁₋₇aminoalkyl (e.g., aminoethyl; 2-(N,N-diethylamino)ethyl; 2-(4-morpholino)ethyl); and acyloxy-C₁₋₇alkyl (e.g., acyloxymethyl; acyloxyethyl; pivaloyloxymethyl; acetoxymethyl; 1-acetoxyethyl; 1-(1-methoxy-1-methyl)ethyl-carbonxyloxyethyl; 1-(benzoyloxy)ethyl; isopropoxy-carbonyloxymethyl; 1-isopropoxy-carbonyloxyethyl; cyclohexyl-carbonyloxymethyl; 1-cyclohexyl-carbonyloxyethyl; cyclohexyloxy-carbonyloxymethyl; 1-cyclohexyloxy-carbonyloxyethyl; (4-tetrahydropyranyloxy) carbonyloxymethyl; 1-(4-tetrahydropyranyloxy)carbonyloxyethyl; (4-tetrahydropyranyl)carbonyloxymethyl; and 1-(4-tetrahydropyranyl)carbonyloxyethyl).

Also, some prodrugs are activated enzymatically to yield the active compound, or a compound which, upon further chemical reaction, yields the active compound (for example, as in ADEPT, GDEPT, LIDEPT, etc.). For example, the prodrug may be a sugar derivative or other glycoside conjugate, or may be an amino acid ester derivative.

Simultaneous or Sequential Administration

Compositions may be administered alone or in combination with other treatments, either simultaneously or sequentially dependent upon the condition to be treated.

In this specification an oncolytic virus and chemotherapeutic agent may be administered simultaneously or sequentially.

Simultaneous administration refers to administration of the oncolytic virus and chemotherapeutic agent together, for example as a pharmaceutical composition containing both agents, or immediately after each other and optionally via the same route of administration, e.g. to the same artery, vein or other blood vessel.

Sequential administration refers to administration of one of the oncolytic virus or chemotherapeutic agent followed after a given time interval by separate administration of the other agent. It is not required that the two agents are administered by the same route, although this is the case in some embodiments. The time interval may be any time interval.

Whilst simultaneous or sequential administration is intended such that both the oncolytic virus and chemotherapeutic agent are delivered to the same tumor tissue to effect treatment it is not essential for both agents to be present in the tumor tissue in active form at the same time.

However, in some embodiments of sequential administration the time interval is selected such that the oncolytic herpes simplex virus and chemotherapeutic agent are expected to be present in the tumor tissue in active form at the same time, thereby allowing for a combined, additive or synergistic effect of the two agents in treating the tumor. In such embodiments the time interval selected may be any one of 5 minutes or less, 10 minutes or less, 15 minutes or less, 20 minutes or less, 25 minutes or less, 30 minutes or less, 45 minutes or less, 60 minutes or less, 90 minutes or less, 120 minutes or less, 180 minutes or less, 240 minutes or less, 300 minutes or less, 360 minutes or less, or 720 minutes or less, or 1 day or less, or 2 days or less.

Cancer

A cancer may be any unwanted cell proliferation (or any disease manifesting itself by unwanted cell proliferation), neoplasm or tumor or increased risk of or predisposition to the unwanted cell proliferation, neoplasm or tumor. The cancer may be benign or malignant and may be primary or secondary (metastatic). A neoplasm or tumor may be any abnormal growth or proliferation of cells and may be located in any tissue. Examples of tissues include the adrenal gland, adrenal medulla, anus, appendix, bladder, blood, bone, bone marrow, brain, breast, cecum, central nervous system (including or excluding the brain) cerebellum, cervix, colon, duodenum, endometrium, epithelial cells (e.g. renal epithelia), gallbladder, oesophagus, glial cells, heart, ileum, jejunum, kidney, lacrimal glad, larynx, liver, lung, lymph, lymph node, lymphoblast, maxilla, mediastinum, mesentery, myometrium, nasopharynx, omentume, oral cavity, ovary, pancreas, parotid gland, peripheral nervous system, peritoneum, pleura, prostate, salivary gland, sigmoid colon, skin, small intestine, soft tissues, spleen, stomach, testis, thymus, thyroid gland, tongue, tonsil, trachea, uterus, vulva, white blood cells.

Tumors to be treated may be nervous or non-nervous system tumors. Nervous system tumors may originate either in the central or peripheral nervous system, e.g. glioma, medulloblastoma, meningioma, neurofibroma, ependymoma, Schwannoma, neurofibrosarcoma, astrocytoma and oligodendroglioma. Non-nervous system cancers/tumors may originate in any other non-nervous tissue, examples include melanoma, mesothelioma, lymphoma, myeloma, leukemia, Non-Hodgkin's lymphoma (NHL), Hodgkin's lymphoma, chronic myelogenous leukemia (CML), acute myeloid leukemia (AML), myelodysplastic syndrome (MDS), cutaneous T-cell lymphoma (CTCL), chronic lymphocytic leukemia (CLL), hepatoma, epidermoid carcinoma, prostate carcinoma, breast cancer, lung cancer, colon cancer, ovarian cancer, pancreatic cancer, thymic carcinoma, NSCLC, haematologic cancer and sarcoma.

A tumor may be a malignant peripheral nerve sheath tumor (MPNST), a sarcoma, solid tumor, bone sarcoma or neuroblastoma.

A tumor may arise during childhood e.g. in a subject under the age of one of 18, 16, 14, 12 or 10 years old. Such tumors are described herein as “pediatric tumors”.

Subjects

The subject to be treated may be any animal or human. The subject is preferably mammalian, more preferably human. The subject may be a non-human mammal, but is more preferably human. The subject may be male or female. The subject may be a patient. A subject may have been diagnosed with a cancer, or be suspected of having a cancer.

The subject may be a child, e.g. under the age of one of 18, 16, 14, 12 or 10 years old. The subject may be an adult who developed their cancer whilst a child.

Other Chemotherapeutic Agents

In addition to treating a cancer by using an oncolytic virus with an Aurora kinase inhibitor or a histone deacetylase (HDAC) inhibitor, subjects being treated may also receive treatment with other chemotherapeutic agents. For example, other chemotherapeutic agents may be selected from:
(i) alkylating agents such as cisplatin, carboplatin, mechlorethamine, cyclophosphamide, chlorambucil, ifosfamide;
(ii) purine or pyrimidine anti-metabolites such as azathiopurine or mercaptopurine;
(iii) alkaloids and terpenoids, such as vinca alkaloids (e.g. vincristine, vinblastine, vinorelbine, vindesine), podophyllotoxin, etoposide, teniposide, taxanes such as paclitaxel (Taxol™), docetaxel;
(iv) topoisomerase inhibitors such as the type I topoisomerase inhibitors camptothecins irinotecan and topotecan, or the type II topoisomerase inhibitors amsacrine, etoposide, etoposide phosphate, teniposide;
(v) antitumor antibiotics (e.g. anthracyline antibiotics) such as dactinomycin, doxorubicin (Adriamycin™), epirubicin, bleomycin, rapamycin;
(vi) antibody based agents, such as anti-VEGF, anti-TNFα, anti-IL-2, antiGpIIb/IIIa, anti-CD-52, anti-CD20, anti-RSV, anti-HER2/neu(erbB2), anti-TNF receptor, anti-EGFR antibodies, monoclonal antibodies or antibody fragments, examples include: cetuximab, panitumumab, infliximab, basiliximab, bevacizumab (Avastin®), abciximab, daclizumab, gemtuzumab, alemtuzumab, rituximab (Mabthera®), palivizumab, trastuzumab, etanercept, adalimumab, nimotuzumab,
(vii) EGFR inhibitors such as erlotinib, cetuximab and gefitinib
(viii) anti-angiogenic agents such as bevacizumab (Avastin®).

Routes of Administration

Viruses, chemotherapeutic agents, medicaments and pharmaceutical compositions according to aspects of the present invention may be formulated for administration by a number of routes, including but not limited to, parenteral, intravenous, intra-arterial, intramuscular, intratumoural and oral. Viruses, chemotherapeutic agents, medicaments and compositions may be formulated in fluid or solid form. Fluid formulations may be formulated for administration by injection to a selected region of the human or animal body.

Dosage Regime

Multiple doses of the oncolytic virus may be provided. One or more, or each, of the doses may be accompanied by simultaneous or sequential administration of a chemotherapeutic agent.

Multiple doses may be separated by a predetermined time interval, which may be selected to be one of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days, or 1, 2, 3, 4, 5, or 6 months.

By way of example, doses may be given once every 7, 14, 21 or 28 days (plus or minus 3, 2, or 1 days). The dose of oncolytic virus given at each dosing point may be the same, but this is not essential. For example, it may be appropriate to give a higher priming dose at the first, second and/or third dosing points.

Kits

In some aspects of the present invention a kit of parts is provided. In some embodiments the kit may have at least one container having a predetermined quantity of oncolytic virus, e.g. predetermined viral dose or number/quantity/concentration of viral particles. The oncolytic virus may be formulated so as to be suitable for injection or infusion to a tumor or to the blood. In some embodiments the kit may further comprise at least one container having a predetermined quantity of chemotherapeutic agent. The chemotherapeutic agent may also be formulated so as to be suitable for injection or infusion to the tumor or to the blood, or alternatively may be formulated for oral administration. In some embodiments a container having a mixture of a predetermined quantity of oncolytic virus and predetermined quantity of chemotherapeutic agent is provided, which may optionally be formulated so as to be suitable for injection or infusion to the tumor or to the blood.

In some embodiments the kit may also contain apparatus suitable to administer one or more doses of the oncolytic virus and/or chemotherapeutic agent. Such apparatus may include one or more of a catheter and/or needle and/or syringe, such apparatus preferably being provided in sterile form.

The kit may further comprise instructions for the administration of a therapeutically effective dose of the oncolytic virus and/or chemotherapeutic agent.

The invention includes the combination of the aspects and preferred features described except where such a combination is clearly impermissible or expressly avoided.

The section headings used herein are for organizational purposes only and are not to be construed as limiting the subject matter described.

Aspects and embodiments of the present invention will now be illustrated, by way of example, with reference to the accompanying figures. Further aspects and embodiments will be apparent to those skilled in the art. All documents mentioned in this text are incorporated herein by reference.

### BRIEF DESCRIPTION OF THE FIGURES

Embodiments and experiments illustrating the principles of the invention will now be discussed with reference to the accompanying figures in which:

FIG. 1. Illustration of selected aurora kinase inhibitors.

FIG. 2. Illustration of selected HDAC inhibitors.

FIG. 3. Chart showing MTS cell survival for HSV1716 in combination with MLN8237 in S462TY cells. 0V=no virus, 0D=no drug, 1V=moi 0.045, 2V=moi 0.45, 1D=30 nM MLN8237, 2D=50 nM MLN8237, 3D=70 nM MLN8237 [the combinations indicated at the right from top to bottom correspond to the bars when read from left to right].

FIG. 4. Chart showing FaCl plot from the data in FIG. 3 for HSV1716 in combination with MLN8237.

FIGS. 5a and 5b. (a) Chart showing tumor growth inhibition by iTu HSV1716 alone, MLN8237 (Alisertib) alone, or the combination in S462TY flank xenografts. Treatment schedules and doses are indicated at the base of the graph. (b) Chart showing tumor growth inhibition by IV HSV1716 alone, MLN8237 (Alisertib) alone, or the combination in S462TY flank xenografts. Treatment schedules and doses are indicated at the base of the graph.

FIG. 6. Chart showing MLN8237 (Alisertib) enhances ITu HSV1716 therapy in mice with S462TY xenografts. Mice bearing S462TY xenograft tumors were treated with MLN8237 and oHSV (n=10). Animals were dosed 2× per day (5 days per week) with MLN8237 (20 mg/kg), or vehicle, by oral gavage at day 0 for 42 days and/or a single ITu injection of HSV1716 (1×10⁷ pfu) or PBS at day 7 (p value for the combination=0.0194 as determined by log-rank (Mantel-Cox) test).

FIG. 7. Chart showing MLN8237 (Alisertib) enhances IV HSV1716 therapy in mice with S462TY xenografts. Mice bearing S462TY xenograft tumors were treated with MLN8237 and oHSV (n=10). Animals were dosed 2× per day (5 days per week) with MLN8237 (20 mg/kg), or vehicle, by oral gavage at day 0 for 42 days and/or a single IV injection of HSV1716 (9×10⁷ pfu) or PBS at day 7 (p value for the combination=0.0033 as determined by log-rank (Mantel-Cox) test).

FIG. 8. Illustration of treatment regimens for each of the four groups of nude mice with S462TY xenografts.

FIG. 9. Chart showing tumor growth inhibition up to day 28 by HSV1716 alone, MLN8237 alone or the combination in S462TY flank xenografts. Groups of mice were treated according to the cyclical schema in FIG. 8 with MLN8237 and HSV1716 administrations indicated by blocks or arrows respectively.

FIG. 10. Chart showing tumor growth inhibition for individual mice by HSV1716 alone, MLN8237 (Alisertib) alone or the combination in S462TY flank xenografts. Groups of mice were treated according to cyclical schema (FIG. 8) with MLN8237 and HSV1716 administrations indicated.

FIG. 11. Chart showing significant tumor growth inhibition by the combination of HSV1716 and MLN8237 compared to HSV1716 alone on day 28 of treatment in S462TY flank xenografts.

FIG. 12. Chart showing MLN8237 (Alisertib) enhances ITu HSV1716 therapy in mice with S462TY xenografts. Survival of control mice or mice treated with HSV1716 alone, MLN8237 alone or the combination in S462TY flank xenografts. Groups of mice were treated according to the cyclical schema indicated with p value determined by log-rank (Mantel-Cox) test.

FIG. 13. Illustration of treatment regimens for each of the four groups of nude mice with SK-N-AS xenografts.

FIG. 14. Chart showing tumor growth inhibition by HSV1716 alone, MLN8237 (Alisertib) alone, or the combination in SK-N-AS flank xenografts. Groups of mice were treated according to the schema in FIG. 13 with MLN8237 and HSV1716 administrations as indicated.

FIGS. 15a to 15d. Chart showing tumor growth in individual control mice (a), or mice treated with HSV1716 alone (b), MLN8237 (Alisertib) alone (c), or the combination (d) in SK-N-AS flank xenografts.

FIG. 16. Chart showing significant tumor growth inhibition by HSV1716 or MLN8237 alone compared to control mice, and by the combination of HSV1716 and MLN8237 compared to HSV1716 or MLN8237 alone on day 21 of treatment in SK-N-AS flank xenografts.

FIG. 17. Chart showing MLN8237 enhances ITu HSV1716 therapy in mice with SK-N-AS xenografts. Survival of control mice or mice treated with HSV1716 alone, MLN8237 alone, or the combination in SK-N-AS flank xenografts. Groups of mice were treated according to the schema in FIG. 13 with p value determined by log-rank (Mantel-Cox) test.

FIG. 18. Chart showing HSV1716 titres at 2 and 48 hours after ITu injection of 1×10⁷ pfu HSV1716 of control or MLN8237-treated mice with S462TY xenografts.

FIG. 19. Chart showing results from FACS analysis of cells extracted from S462TY xenografts 3 days after ITu injection of 1×10e7 pfu HSV1716 to identify CD45.2+ and CD11b+ leukocytes, neutrophils (Neu), natural killer cells (NK), tumor-associated macrophages (TAM), myeloid-derived suppressor cells (MDSC) and B-cells. Mice were treated with either PBS, MLN8237 (MLN) or ITu HSV1716 alone or HSV1716 in combination with MLN8237 (MLN+HSV1716).

FIG. 20. Chart showing MLN8237 (Alisertib) significantly increases intracellular HSV1716 genomes 30 min post infection of S462TY cells.

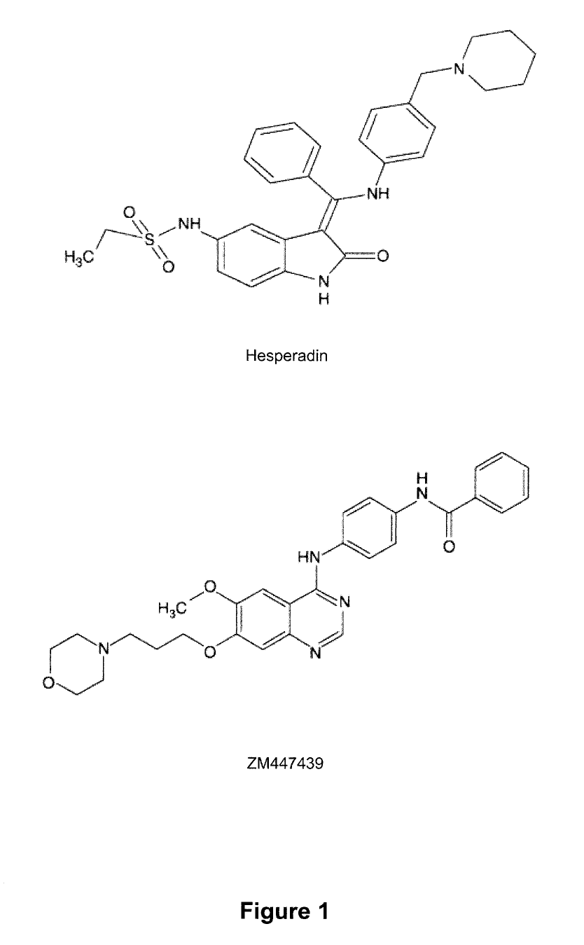

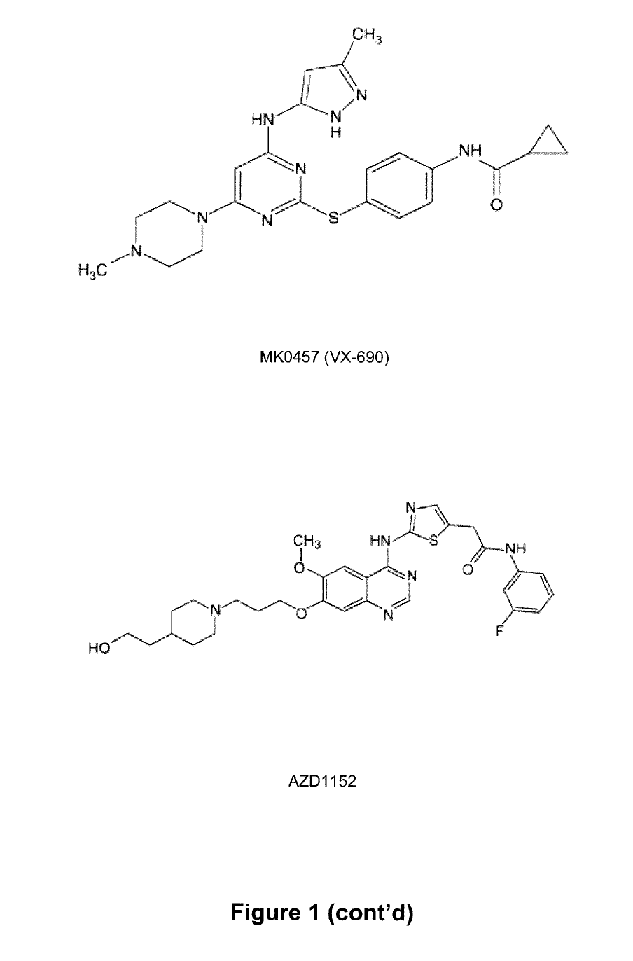

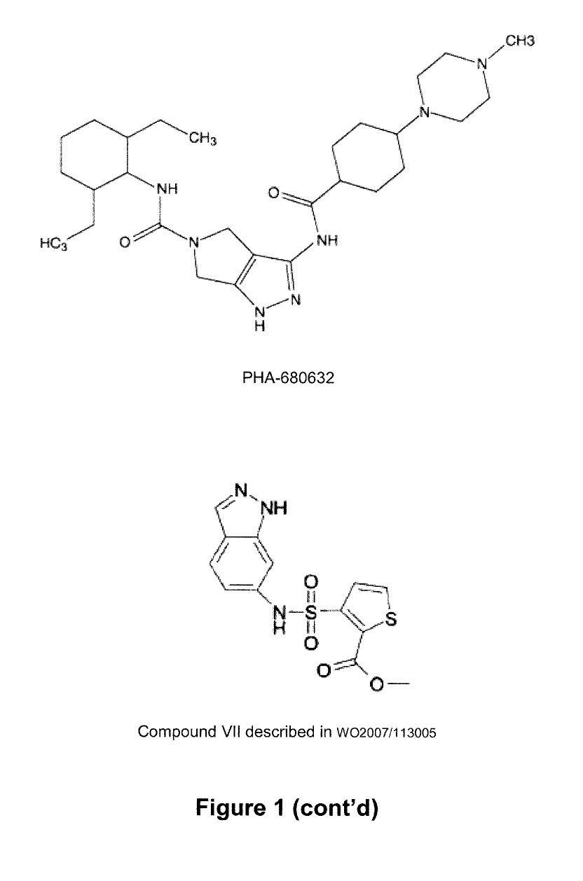

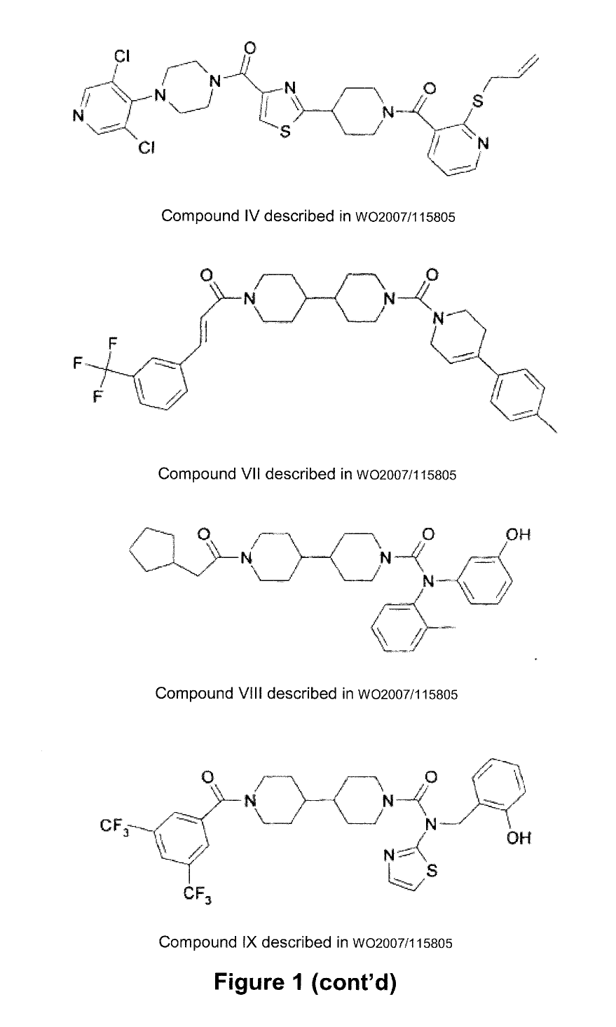

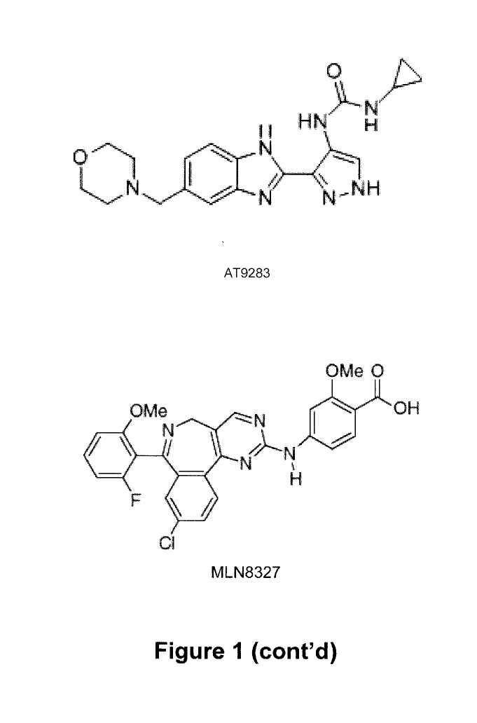

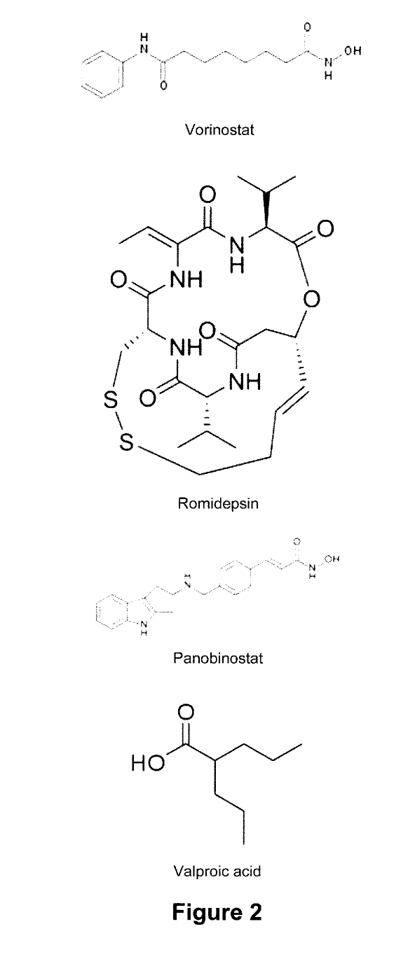

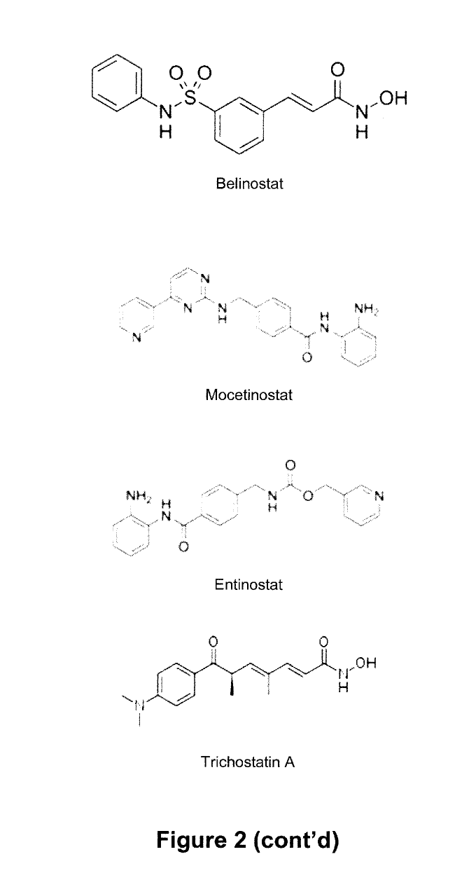

### DETAILED DESCRIPTION OF THE INVENTION

The details of one or more embodiments of the invention are set forth in the accompanying description below including specific details of the best mode contemplated by the inventors for carrying out the invention, by way of example. It will be apparent to one skilled in the art that the present invention may be practiced without limitation to these specific details.

Aurora kinase inhibitors and HDAC inhibitors are reported to arrest tumor growth. We realise that by providing an attenuation in tumor growth an oncoytic virus will have an improved opportunity to lyse tumor cells and successfully destroy the tumor.

### EXAMPLES

### Example 1

PURPOSE: To evaluate the therapeutic effect of novel combination therapy against human tumor cell lines using oncolytic herpes simplex virus (oHSV) and chemotherapy using MTS Cell Viability assays.

Methods

Cell Lines

Human tumor cell lines are grown and cultured in complete media (Dulbecco's Modified Eagle Medium (DMEM) supplemented with 10% Fetal Bovine Serum (FBS)) and incubated at 37° C. and 5% CO₂.

MTS Cell Viability Assays

Cells are counted using a hemocytometer, and added to 96 well plates at a concentration of 500 cells/well in 50 μL complete media. Following 24 hour incubation, cells were treated with either a single agent virus, a small molecule signal transduction inhibitor, or in combination.

Viral Studies

Single agent virus MTS assays are performed over a range of multiplicities of infection (MOI) (0.001, 0.01, 0.10, 1.0) in the cell lines. Cells are infected with HSV1716 and read at days 0, 1, 2, 4, and 6 post-infection. Viral MTS assays are completed by the addition of 20 μL of MTS dye to each well, followed by a 4 hour incubation period before adding 25 μL of Sodium Dodecyl Sulfate (SDS). Each plate is read by a 96 well plate spectrophotometer at a wavelength of 490 nm.

MTS Combination Studies

Viral combination studies are carried out using HSV1716 in the tumor cell lines at a single time point (day 4 post-infection). Chemotherapy agents are added in combination with virus at the inhibition concentration (IC) that caused 25, 50 and 75% decrease of cell viability as a single agent as established in previous experiments. The data is analyzed and synergy is determined via Chou-Talalay analysis. MTS assays and Chou-Talalay analysis are techniques that are well-known in the art.

Results

HSV1716 reduces the survival of tumor cell growth in vitro as monotherapy and in combination with aurora kinase inhibitors and/or HDAC inhibitors.

### Example 2

### In Vitro and In Vivo Combination Studies with HSV1716 and the Aurora Kinase A Inhibitor MLN8237 in Malignant Peripheral Nerve Sheath Tumors (MPNSTs) and Neuroblastoma Cell Lines

MLN8237 (Alisertib)

MLN8237 (Takeda/Millennium) is a second-generation Aurora Kinase A inhibitor. It inhibits Aurora Kinase A with an IC50 of 1 nM in biochemical assays and has 200-fold selectivity for Aurora Kinase A over Aurora Kinase B in cell assays. A broad screen of receptors and ion channels showed no significant cross-reactivity. The compound blocks the growth of multiple tumor cell lines with GI50 values as low as 16 nM. Growth inhibition is associated with mitotic spindle abnormalities, accumulation of cells in mitosis, polyploidy, and apoptosis. It is orally available and rapidly absorbed. At effective doses a transient inhibition of histone H3 phosphorylation is observed (consistent with Aurora Kinase B inhibition being dominant) followed by marked elevation of histone H3 phosphorylation (consistent with Aurora Kinase A inhibition being dominant). Maximum in vivo efficacy, in multiple xenografts, has been achieved with oral doses of 20 mg/kg given twice a day for 21 consecutive days, although other regimens are also effective (Dar et al., Aurora Kinase Inhibitors—Rising Stars in Cancer Therapeutics? Mol Cancer Ther. (2010) 9; 268.).

Malignant Peripheral Nerve Sheath Tumors

Malignant peripheral nerve sheath tumors (MPNSTs) are sarcomas which originate from peripheral nerves or from cells, such as Schwann cells, perineural cells, or fibroblasts, associated with the nerve sheath. Diagnosis and classification can be difficult. In general, a sarcoma arising from a peripheral nerve or a neurofibroma is considered to be a MPNST. The term MPNST replaces a number of previously used names including malignant schwannoma, neurofibrosarcoma, and neurogenic sarcoma. MPNSTs comprise approximately 5-10% of all soft tissue sarcomas, occurring either spontaneously or in association with neurofibromatosis-1 (NF1).

Surgical resection offers the best outcome with respect to both local recurrence and distant metastases for MPNST. Radiation therapy is an integral part of local disease control in most soft tissue sarcomas. Together with wide surgical excision, radiation therapy offers local and overall survival rates which are similar to those following amputation. Treatment of soft-tissue sarcomas with adjuvant radiation therapy has yielded a statistically significant reduction in the rates of local disease recurrence but has not however had a meaningful reduction in either rates of distant metastases or overall survival (Vraa et al., (1998) Prognostic factors in soft tissue sarcomas: the Aarhus experience. Eur J Cancer, 34((12)): 1876-82, Yang et al., (1998) Randomized prospective study of the benefit of adjuvant radiation therapy in the treatment of soft tissue sarcomas of the extremity. J Clin Oncol, 16((1)): 197-203).

Overall survival with MPNSTs is poor, and the usual chemotherapy used for soft-tissue sarcomas does not improve the outcome (Pervaiz et al., (2008) A systematic meta-analysis of randomized controlled trials of adjuvant chemotherapy for localized resectable soft-tissue sarcoma. Cancer; 113(3):573-581). The rarity of MPNST means that consistent data regarding chemotherapy sensitivity are lacking; no phase II or III trials were carried out specifically in MPNST.

Soft Tissue Sarcomas in Childhood

Soft tissue sarcomas are common solid tumors in children less than 20 years old, after brain tumors, lymphomas, and carcinomas (mainly thyroid and melanoma). As in adult patients, pediatric patients experience a wide variety of sarcomas. The most common soft tissue sarcoma in children is rhabdomyosarcoma. Over the past several decades, advances in the combined use of chemotherapy, surgery and radiation have markedly improved the survival of patients with localized soft tissue sarcomas from <25% to nearly 70% [Pappo et al. (1995). Biology and therapy of pediatric rhabdomyosarcoma. Journal of Clinical Oncology 13: 2123-2139.]. There remain a number of challenges, however, in treating patients with rhabdomyosarcoma. Metastatic disease is the major predictor of poor outcome and has not been significantly impacted by combination therapy.

Bone sarcomas are the third most common cancer in adolescence, occurring less frequently than only lymphomas and brain tumors [Ries et al. (1999). Cancer incidence and survival among children and adolescents: United States SEER Program 1975-1995, National Cancer Institute, SEER Program. NIH Pub. No. 99-4649. Bethesda, Md.]. The two most common types in children and young adults are osteosarcoma and Ewing sarcoma. The mainstay of therapy for both types is surgery, and limb-sparing procedures can usually be used to preserve function. These procedures, however, may be fraught with significant morbidity in terms of recovery and ultimate function. Chemotherapy also is required to treat micrometastatic disease, which is present but not detectable in most patients at diagnosis. Radiation therapy is also used as an important treatment for Ewing sarcoma. Radiation therapy is essentially not an option for patients with osteosarcomas because they are uniformly resistant to radiation. For both tumor types, cure rates with combination therapies for localized disease are in the range of 60-70% [Ries et al. (1999). Cancer incidence and survival among children and adolescents: United States SEER Program 1975-1995, National Cancer Institute, SEER Program. NIH Pub. No. 99-4649. Bethesda, Md.]. Patients who present with metastases or with multifocal disease, however, have a very poor prognosis with long-term survival rates<30%.

Neuroblastoma (Nb) is the most common extracranial solid tumor in children<10 years old. The median age of diagnosis is 17.3 months, with 40% of patients diagnosed as infants, 90% at ages younger than 5 years and 97.8% by 10 years of age [Olshan, A., and Bunin, G. (2000). Epidemiology of Neuroblastoma. In Neuroblastoma (G. Brodeur, T. Sawada, Y. Tsuchida and P. Voute, Eds.), pp. 33-39. Elsevier Science, Amsterdam, Brodeur, G., and Maris, J. (2001). Neuroblastoma. In Principles and Practice of Pediatric Oncology (P. Pizzo and D. Poplack, Eds.), pp. 896-937. Lippincott Williams & Wilkins, Philadelphia.]. Unfortunately, the long term survival rate of patients with high-risk neuroblastoma is very poor. The risk factors that characterize a poor prognosis include age of more than one year, higher stage of the disease, MYCN amplification and non-favorable histology. Encouragingly, there has been an improvement in patient survival of high-risk neuroblastoma through the use of high dose chemotherapy, autologous stem cell rescue and retinoic acid [Matthay et al. (1999). Treatment of high-risk neuroblastoma with intensive chemotherapy, radiotherapy, autologous bone marrow transplantation, and 13-cis-retinoic acid. Children's Cancer Group. New England Journal of Medicine 341: 1165-1173.]. Nevertheless, survival remains<50%, particularly if tumors have MYCN amplification, which correlates with early progression [Seeger et al. (1985). Association of multiple copies of the N-myc oncogene with rapid progression of neuroblastomas. New England Journal of Medicine 313: 1111-1116.].

The use of combined approaches with surgery, chemotherapy and radiation has resulted in improved patient survival rates for all of the tumors described above to varying degrees. However, the level of maximum benefits with these modalities have essentially been reached.

A: HSV1716 in Combination with the Aurora Kinase Inhibitor MLN8237 in the Malignant Peripheral Nerve Sheath Tumor Cell Line S462TY and the Neuroblastoma Cell Line SK-N-AS.

In Vitro Combination—Chou/Talalay Analysis.

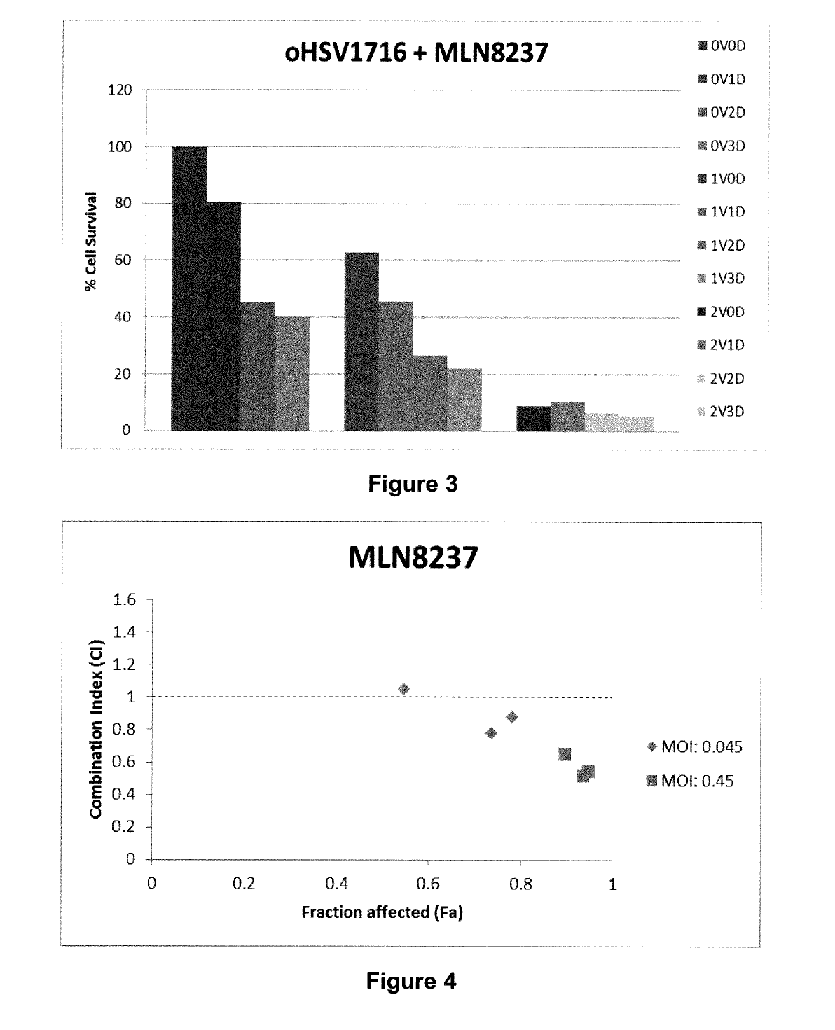

HSV1716 in combination with MLN8237 was assessed in the malignant peripheral nerve sheath tumor (MPNST) cell line S462TY. Toxicity was measured using MTS cell survival assay at 0, 30, 50 or 70 nM MLN8237 in combination with HSV1716 at moi 0, 0.045 or 0.45 and the results analysed for synergistic interactions by the Chou/Talalay method. MTS cell survival profiles are shown in FIG. 3 and the resulting FaCl plot from the Chou/Talalay analysis of this data is shown in FIG. 4. HSV1716 combines with MLN8237 synergistically to enhance cell killing as 5/6 combinations generated CI<1 (Table 2).

TABLE 2
Fa and Cl values from Chou/Talalay analysis of HSV1716
in combination with MLN8237.

| MLN8237 Dose (nM) | HSV1716 MOI | Fa | Cl |
|---|---|---|---|
| 30 | 0.045 | 0.544767 | 1.054598 |
| 50 | 0.045 | 0.734438 | 0.780505 |
| 70 | 0.045 | 0.779835 | 0.882132 |
| 30 | 0.45 | 0.895564 | 0.654121 |
| 50 | 0.45 | 0.934083 | 0.519477 |
| 70 | 0.45 | 0.945432 | 0.548158 |

In Vivo Combination of HSV1716 and MLN8237

a) HSV1716 and MLN8237 in a S462TY Nude Mouse Xenograft Model

The S462TY cell line readily formed flank xenografts in nude mice and a 6-arm in vivo efficacy study (n=10/group) was performed for single intravenous or intratumoral injections of HSV1716 in combination with orally administered MLN8237. MLN8237 at 20 mg/kg or its carrier vehicle were administered by oral gavage twice daily for 5 days per week from day 0 for 42 days. Single intratumoral (ITu) injections of 1.0×10⁷ pfu HSV1716 or PBS or single intravenous (IV) injections of 9.0×10⁷ pfu HSV1716 or PBS were administered on day 7 and tumor growth and survival were monitored.

The six groups of mice were:—
Control (vehicle+ITu PBS)
MLN8237 (+ITu PBS)
HSV1716 (ITu)+vehicle
HSV1716 (IV)+vehicle
HSV1716 (ITu)+MLN8237
HSV1716 (IV)+MLN8237

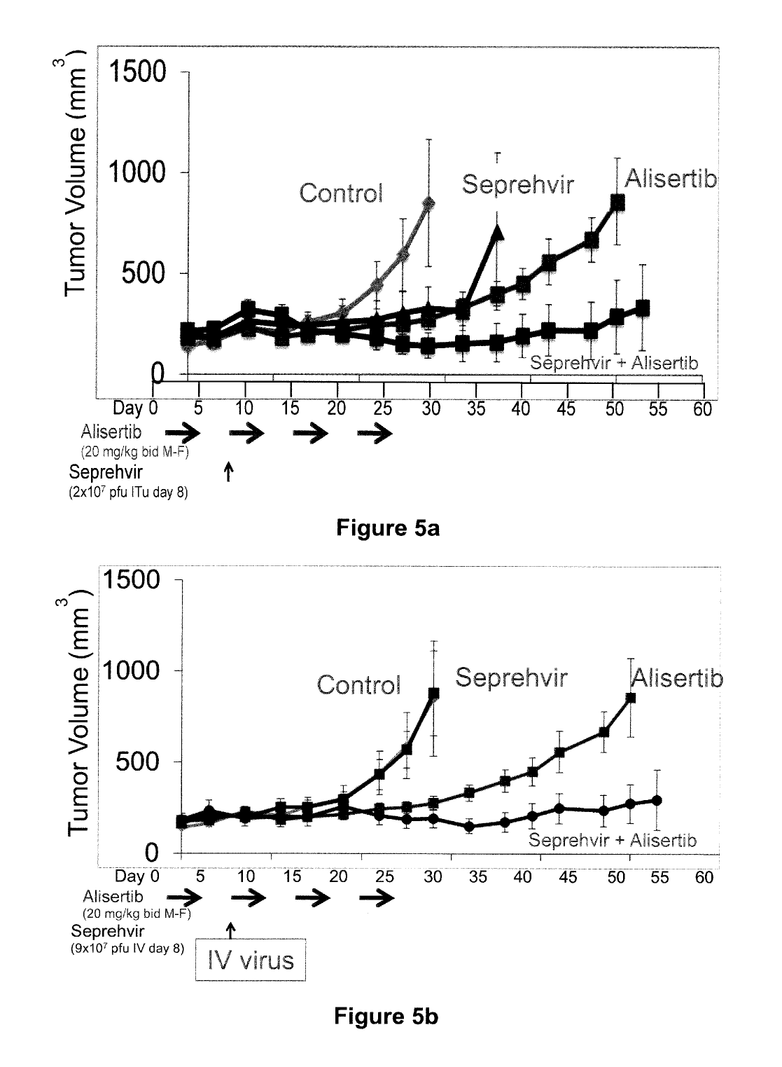

A single intratumoral dose of HSV1716 (FIG. 5a) reduced the growth of the S462TY xenografts in nude mice compared to control treated mice (vehicle+ITu PBS) although the single IV dose of HSV1716 had no effect on tumor growth (FIG. 5b). Two times daily dosing with MLN8237 was initially effective at preventing growth of the xenografts but from day 30 onwards tumor growth was observed and this increased when MLN8237 treatment was stopped on day 42 (FIG. 5a a & b). The combination of single dose HSV1716 either by ITu (FIG. 5a) or IV (FIG. 5b) with twice daily dosing of MLN8237 was highly effective at preventing tumor growth up until day 50 with evidence of some tumor growth thereafter. For the evaluable mice, the “clinical” outcomes for the combination of ITu HSV1716+MLN8237 were 1 progressive disease, 4 partial responses and 3 complete responses (Table 3).

The effects of the single dose IV HSV1716 in combination with MLN8237 are particularly striking as IV HSV1716 alone displayed no tumor growth inhibition (FIG. 5b). For the evaluable mice, the “clinical” outcomes for the combination of IV HSV1716+MLN8237 were 2 progressive disease, 2 stable disease and 3 partial responses (Table 3).

TABLE 3
Clinical responses for evaluable mice treated with ITu HSV1716 alone, IV
HSV1716 alone, MLN8237 alone, or the combinations in S462TY flank
xenografts. Progressive Disease (PD), Stable Disease (SD), Partial
Response (PR) and Complete Response (CR) are responses seen at
day 53 (11 days after last treatment). SD→PD and PR→PD included
the best responses observed for at least 21 days during the 42 day
treatment cycle with the last response observed at day 53.

| | PD | SD-->PD | SD | PR-->PD | PR | CR |
|---|---|---|---|---|---|---|
| Vehicle + ITu PBS | 9 | 0 | 0 | 0 | 0 | 1 |
| MLN8237 + ITu PBS | 4 | 6 | 0 | 0 | 0 | 0 |
| Vehicle + ITu HSV | 2 | 3 | 0 | 2 | 1 | 1 |
| Vehicle + IV HSV | 9 | 0 | 0 | 0 | 0 | 0 |
| MLN8237 + ITu HSV | 0 | 1 | 0 | 0 | 4 | 3 |
| MLN8237 + IV HSV | 0 | 2 | 2 | 0 | 3 | 0 |

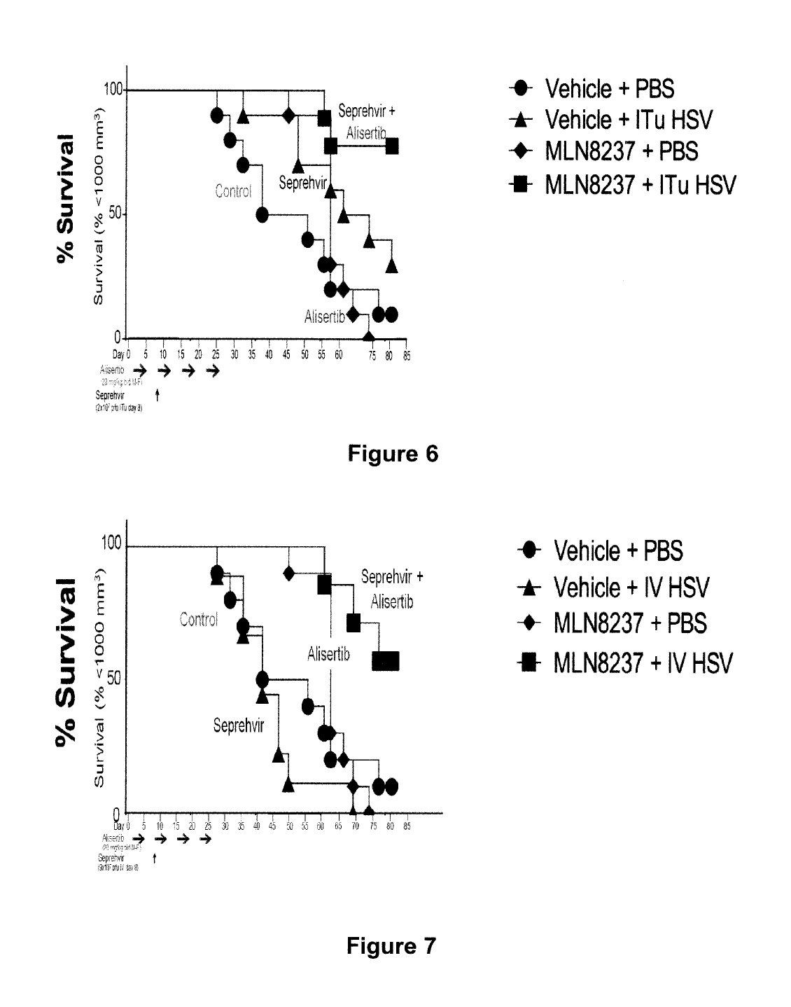

Single ITu injection of HSV1716 or twice daily dosing of MLN8237 improved survival compared to vehicle/PBS-treated controls but the combination had a dramatic and highly significant improvement to survival (FIG. 6). Most of the control or single treatment mice were sacrificed by day 60, at which time the majority of combination treated mice were still alive and remained so thereafter. Single IV administration of HSV1716 did not improve survival compared to control treated mice but again, the combination of single IV HSV1716 with MLN8237 had a dramatic and highly significant improvement to survival (FIG. 7). Most of the mice receiving IV HSV1716 had been sacrificed by day 50, most of those receiving twice daily doses of MLN8237 had been sacrificed by day 60 at which time the majority of the combination treated mice were still alive. Thus the combination of HSV1716 with MLN8237 is highly efficacious in a nude mouse MPNST xenograft model with effective tumor growth inhibition and significantly extended survival.

B: HSV1716 and MLN8237 in a S462TY Nude Mouse Xenograft Model Using Multiple Cycles of Lower Dose MLN8237 Plus HSV1716.

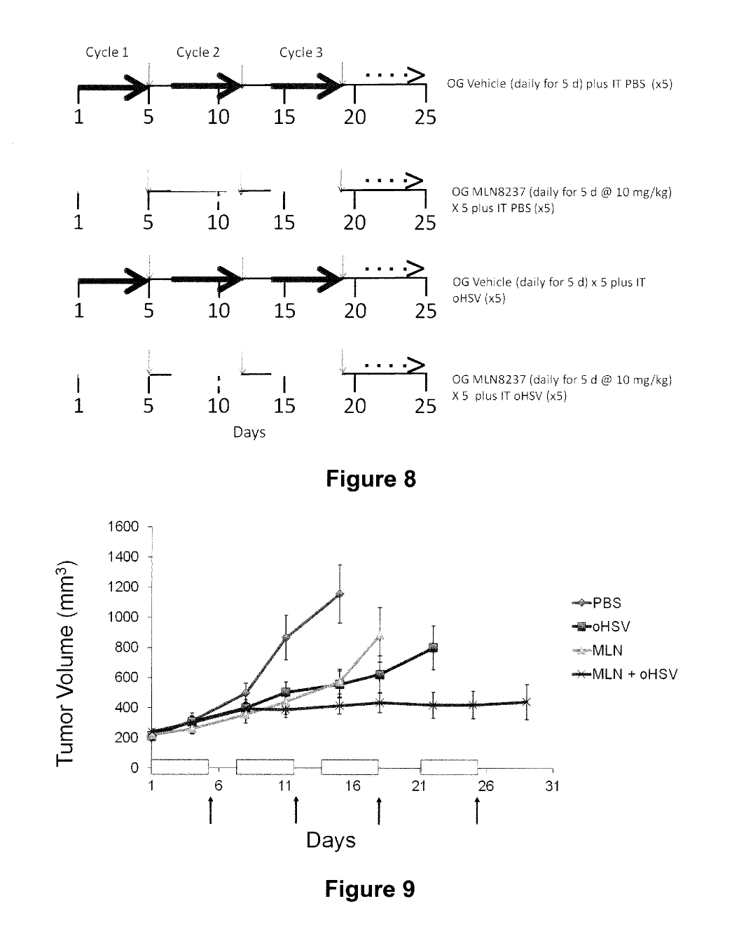

S462TY xenografts were formed in nude mice and a 4-arm in vivo efficacy study (n=10/group) was performed for intratumoral injections of HSV1716 in combination with orally administered MLN8237. MLN8237 at 10 mg/kg or its carrier vehicle were administered by oral gavage once daily for 5 days on five separate occasions with a two day non-treatment interval during which single intratumoral (ITu) injections of 1×10⁷ pfu HSV1716 or PBS were administered. The schema used for each of the 4 groups is shown in FIG. 8 and tumor growth and survival were monitored throughout the experiment.

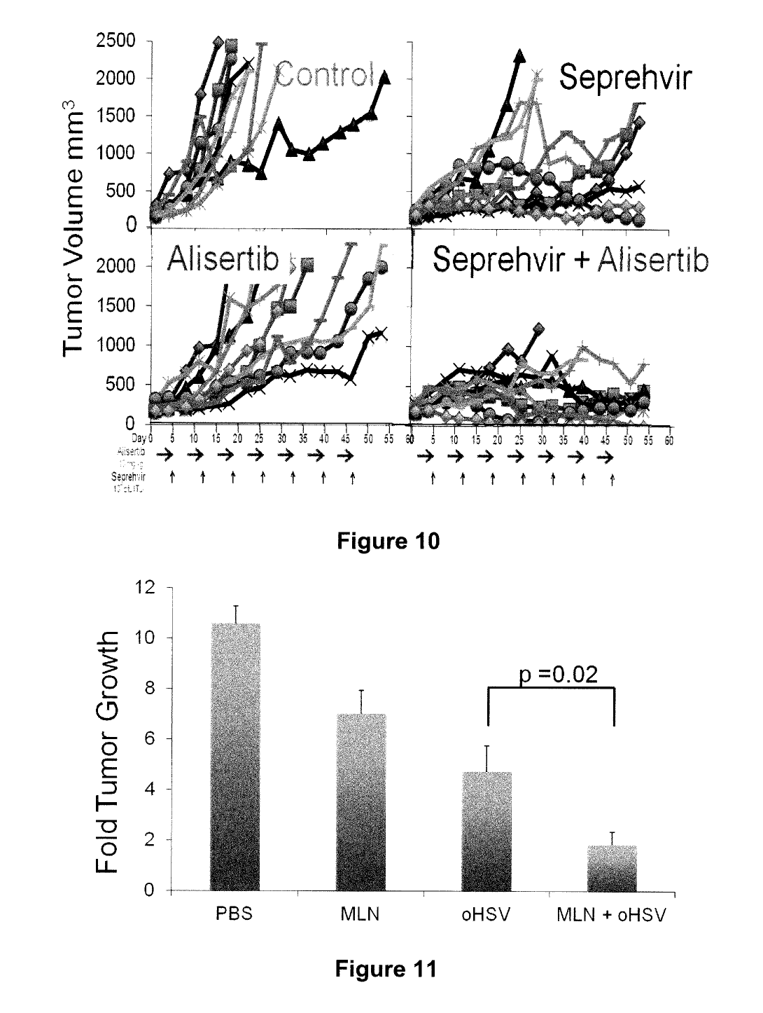

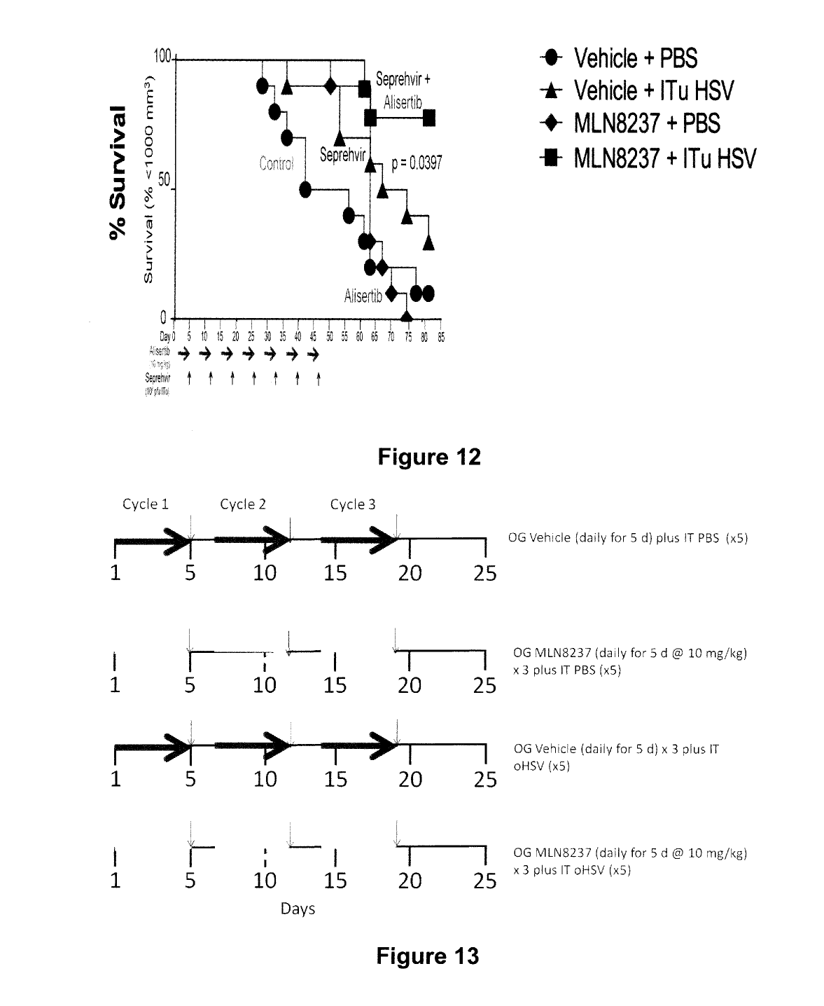

Multiple intratumoral doses of HSV1716 at weekly intervals reduced the growth of the S462TY xenografts in nude mice compared to control-treated mice (vehicle+ITu PBS) (FIG. 9). Daily dosing with 10 mg/kg MLN8237 for 5 days on multiple occasions with a two day non-treatment interval in between also reduced the growth of the S462TY xenografts although, from day 12 onwards, tumor growth was observed in both the MLN8237 and HSV1716 only groups (FIG. 9). The combination of multiple ITu dose of HSV1716 with multiple 5 day, single daily dosing of 10 mg/kg MLN8237 was highly effective at preventing tumor growth as shown for individual mice (FIG. 10) with comparison between the four groups on day 28 indicating a highly significant reduction in tumor growth for the combination treatment (FIG. 11). The multiple dosing combination of HSV1716 with MLN8237 significantly improved survival compared to vehicle/PBS-treated controls and groups treated with single agents only (FIG. 12). Most of the control or single treatment mice were sacrificed by day 30, at which time the majority of combination treated mice were still alive and remained so thereafter. Thus, the combination of HSV1716 with MLN8237 is highly efficacious in a nude mouse MPNST xenograft model with effective tumor growth inhibition and significantly extended survival.

b) HSV1716 and MLN8237 in a SK-N-AS Nude Mouse Xenograft Model

SK-N-AS cells were derived from a bone marrow metastasis located in the brain of a child with poorly differentiated embryonal neuroblastoma and the cell line readily formed flank xenografts in nude mice. A 4-arm in vivo efficacy study (n=10/group) was performed for intratumoral injections of HSV1716 in combination with orally administered MLN8237. MLN8237 at 10 mg/kg or its carrier vehicle were administered by oral gavage once daily for 5 days on five separate occasions with an intervening two day non-treatment interval during which intratumoral (ITu) injections of 1×10⁷ pfu HSV1716 or PBS were administered. The schema used for each of the 4 groups is shown in FIG. 13 and tumor growth and survival were monitored throughout the experiment.

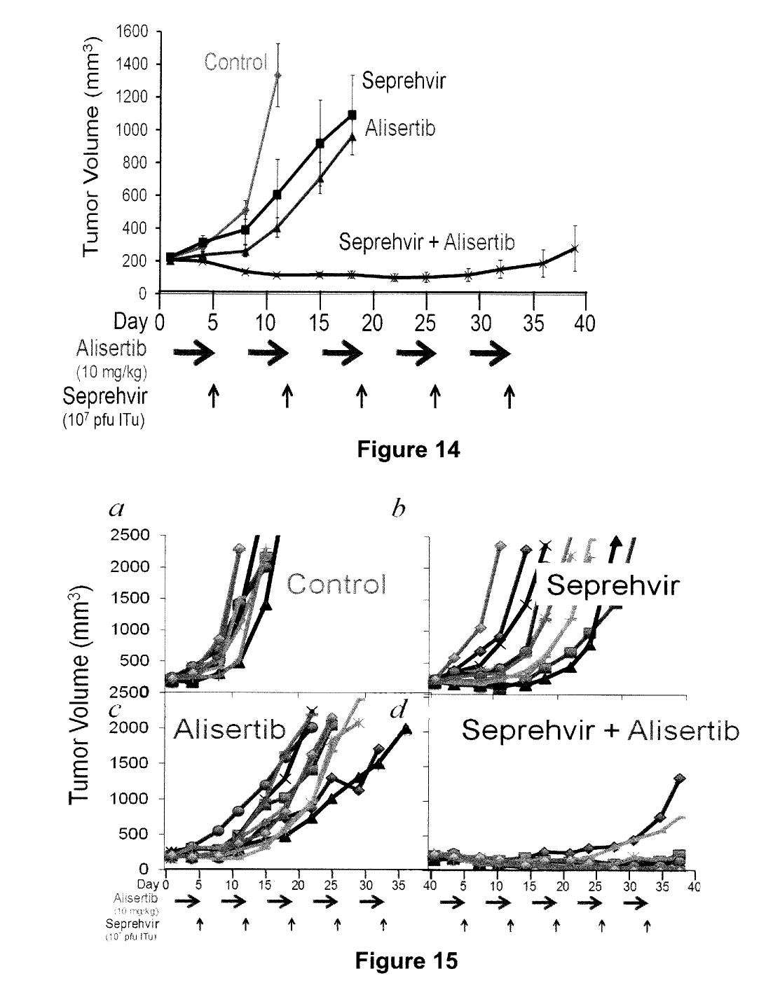

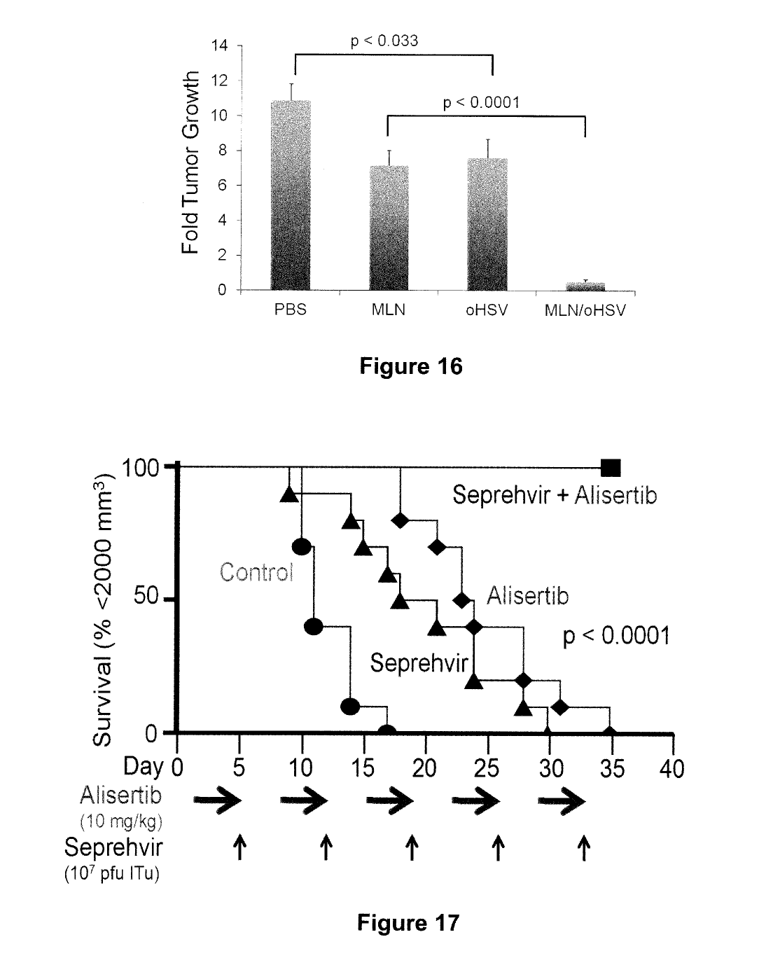

Five intratumoral doses of HSV1716 at weekly intervals reduced the growth of the SK-N-AS xenografts in nude mice compared to control-treated mice (vehicle+ITu PBS) (FIG. 14). Daily dosing with 10 mg/kg MLN8237 for 5 days on five occasions with a two day non-treatment interval in between also reduced the growth of the SK-N-AS xenografts although, from day 10 onwards, tumor growth was observed in both the MLN8237 and HSV1716 only groups (FIG. 14). The combination of multiple ITu doses of HSV1716 with multiple 5 day, single daily dosing of 10 mg/kg MLN8237 was highly effective at preventing tumor growth (FIG. 14) and there is evidence of tumor regression. This highly potent tumor growth inhibition by HSV1716 combined with MLN8237 is clearly observed by comparing the effects on tumor growth in individual mice for the control group (FIG. 15a), and for mice treated with iTu HSV1716 (FIG. 15b), MLN8237 (FIG. 15c) or the combination (FIG. 15d). Comparison between the four groups on day 21 indicated a highly significant reduction in tumor growth for both HSV1716 and MLN8237 alone and for the combination treatment compared to either agent alone (FIG. 16).

The combination of HSV1716 with MLN8237 significantly improved survival compared to vehicle/PBS-treated controls and groups treated with single agents only (FIG. 17). Most of the control mice had been sacrificed by day 14, approximately 50% of mice receiving HSV1716 or MLN8237 alone were still alive on day 25 compared to 100% of the combination treated mice on day 36. This experiment is still ongoing. The current “clinical” evaluation for the treated mice is 2 progressive disease, 4 stable disease, 2 partial responses and 2 complete responses. Thus the combination of HSV1716 with MLN8237 is highly efficacious in a nude mouse neuroblastoma SK-N-AS xenograft model with effective tumor growth inhibition and regression and cures.

c) Mechanism of Action Studies.

The mechanism of action for the potently enhanced efficacy of the MLN8237/HSV1716 combination was investigated in a series of experiments. The combination induces a massive necrosis of tumor cells compared to either agent alone as shown by immunohistochemistry with Ki67 staining (data not shown).

The series of experiments were designed to address the potential mechanisms for the superior efficacy of two agents in combination as follows:
1. MLN8237-induced tumor stasis allows HSV1716 to propagate more efficiently so that tumor no longer grows faster than virus can spread
2. MLN8237+HSV1716 alter the mechanism of cell death
3. MLN8237 causes senescence-induced secretory phenotype resulting in increased recruitment of macrophages that phagocytose dying cells
4. MLN8237 modulates HSV1716-induced innate immune cellular infiltration
5. MLN8237 increases virus susceptibility, permissivity and/or persistence

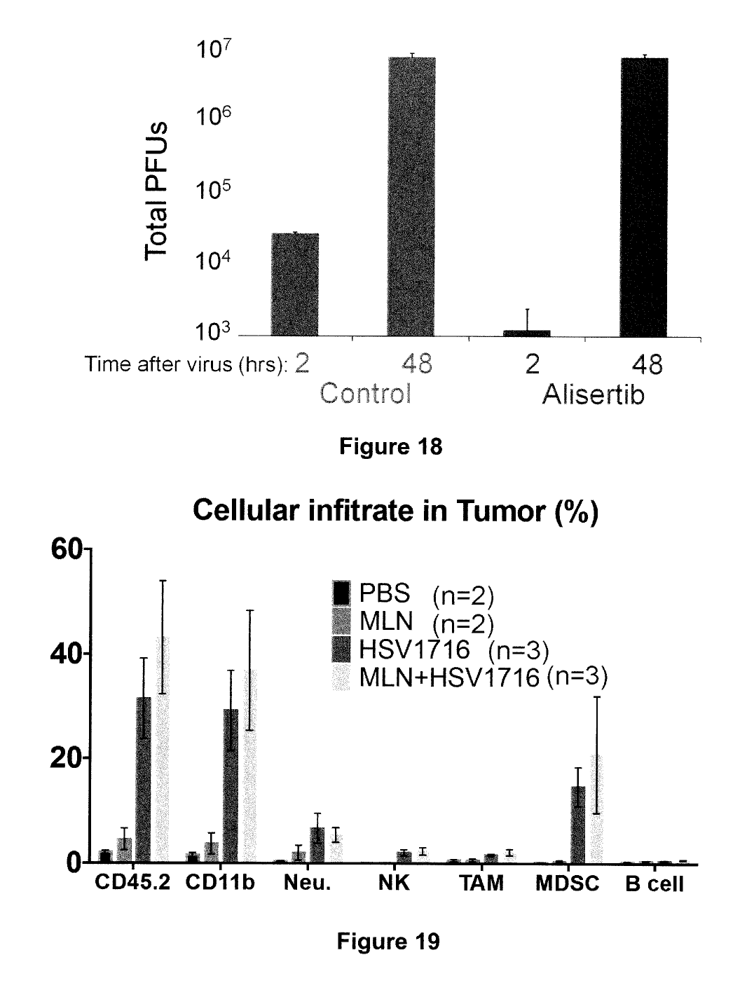

1) Initial experiments suggest that MLN8237 did not improve HSV1716 replication. MLN8237-treated or control, untreated mice with S462TY xenografts received ITu HSV1716 and tumours, removed at 2 and 48 hrs after virus administration, were titrated (FIG. 18). There was no apparent increase in virus titre in the MLN8237-treated compared to the untreated control tumors at 48 hrs. Interestingly, there was less of the free input virus detected at 2 hrs in the MLN8237-treated tumours.

2) Immunohistochemistry for cleaved caspase 3 in S462TY xenografts from control, untreated and HSV1716-, MLN8237- and HSV1716/MLN8237-treated mice suggests that there was no apparent change in levels of apoptosis by the combination treatment compared to either HSV1716 or MLN8237 treatment alone. Sections were prepared at 24 hrs, 72 hrs or 1 week after HSV1716 administration.

3) MLN8237 has been shown to induce a NF-kB-mediated senescence-associated secretory phenotype in melanoma (Liu et al., 2013. EMBO Mol Med 5:149) and potentially, in combination with HSV1716, there is an increase in the numbers of infiltrating macrophages phagocytosing the dead and dying cells. Senescence was detected using X-gal which is a substrate for senescence-associated beta-galactosidase (SA-β-gal or SABG). However, there was little evidence of MLN8237-induced senescence in S462TY xenografts with only 1/18 sections staining after incubation with X-gal (not shown) and there was no apparent increase in the infiltration of activated macrophages (IBa1+) in xenografts from combination-treated mice.

4) FACS analysis was performed on cells extracted from S462TY xenografts to identify innate immune cell infiltration following treatment with HSV1716 alone, MLN8237 alone or the combination of HSV1716 and MLN8237. Cells were extracted 3 days after ITu injection of 1×10e7 pfu HSV1716 and FACS analysis performed to identify CD45.2+ and CD11b+ leukocytes, neutrophils, natural killer (NK) cells, tumor-associated macrophages (TAM), myeloid derived suppressor cells (MDSC) and B-cells (FIG. 19). HSV1716 alone caused increased infiltration of CD45.2+ and CD11b+ leukocytes, neutrophils and MDSC but the combination of HSV1716 with MLN8237 did not cause any significant changes in the recruitment of these or any other innate immune cells.

5) Immunohistochemistry with an anti-HSV antiserum suggests that MLN8237 hugely enhances the initial uptake/infection rate of HSV1716. S462TY xenograft sections from mice treated with HSV1716+vehicle or HSV1716+MLN8237 indicated a vast difference in viral gene expression at the initial 24 hrs time point. There were only several small foci of HSV1716 protein production 24 hrs after ITu injection of 1×10e7 pfu HSV1716 in mice pre-treated with vehicle only whereas at least 50% of cells express viral antigens 24 hrs after ITu injection when mice were pre-treated with MLN8237. This greatly augmented uptake/rate of infection at early times leads to a much improved spread and proliferation at 72 hrs resulting in a much higher rate of tumor cell death and therefore enhanced tumor growth control by the combination.

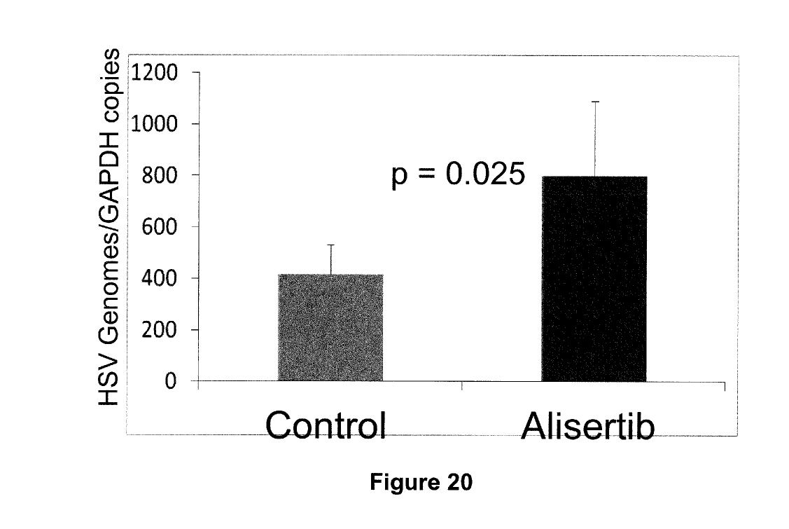

MLN8237 improved the uptake of HSV1716 by S462TY cells in vitro as assessed by quantitative PCR for virus genomes. S462TY cells were infected with HSV1716 and an acid wash after 30 mins was used to remove residual free virus which had not penetrated intracellularly. Q-PCR for HSV genomes was normalised using GAPDH copy number and indicated a significantly higher uptake/infection rate in the presence of MLN8237 (FIG. 20).

Interestingly, this may explain the result presented in FIG. 18 which shows considerably less free virus was detected 2 hrs after ITu injection of HSV1716 in MLN8237-treated mice. However, in the same experiment, levels of titratable HSV1716 were similar+/−MLN8237 and it is possible that MLN8237 promotes cell-to cell spread (not detected by titration) rather than lytic release.

### CONCLUSIONS

HSV1716 and MLN8237 combine synergistically in the MPNST cell line S462TY

Combination of HSV1716 with MLN8237 is highly efficacious in a nude mouse MPNST xenograft model with effective tumor growth inhibition and significantly extended survival.

Mechanism of action studies suggest that MLN8237 improves the initial uptake/rate of infection of HSV1716

Combination of HSV1716 with MLN8237 is highly efficacious in a nude mouse neuroblastoma xenograft model with effective tumor growth inhibition, tumor regression and cures.

1. An oncolytic virus for use in a method of treating cancer, the method comprising simultaneous or sequential administration of an oncolytic virus and an aurora kinase inhibitor.

2. An oncolytic virus for use in a method of treating cancer according to claim 1, wherein the oncolytic virus is an oncolytic herpes simplex virus.

3. An oncolytic virus for use in a method of treating cancer according to claim 2, wherein all copies of the ICP34.5 gene in the genome of the oncolytic herpes simplex virus are modified such that the ICP34.5 gene is incapable of expressing a functional ICP34.5 gene product.

4. An oncolytic virus for use in a method of treating cancer according to claim 2 or 3, wherein the oncolytic herpes simplex virus is a mutant of HSV-1 strain 17.

5. An oncolytic virus for use in a method of treating cancer according to claim 2 or 3, wherein the oncolytic herpes simplex virus is HSV1716.

6. An oncolytic virus for use in a method of treating cancer according to claim 1, wherein the oncolytic virus is selected from one of: an oncolytic reovirus, an oncolytic vaccinia virus, an oncolytic adenovirus, an oncolytic Coxsackie virus, an oncolytic Newcastle Disease Virus, an oncolytic parvovirus, an oncolytic poxvirus, an oncolytic paramyxovirus.

7. Use of an oncolytic virus in the manufacture of a medicament for use in a method of treatment of cancer, wherein the method of treatment comprises administering an aurora kinase inhibitor to the patient in need of treatment.

8. Use of an oncolytic virus according to claim 7, wherein the oncolytic virus is an oncolytic herpes simplex virus.

9. A pharmaceutical composition comprising an oncolytic virus and an aurora kinase inhibitor.

10. A pharmaceutical composition according to claim 9, wherein the oncolytic virus is an oncolytic herpes simplex virus.

11. A pharmaceutical composition according to claim 10, wherein the oncolytic herpes simplex virus is a mutant of HSV-1 strain 17.

12. A pharmaceutical composition according to claim 9, wherein the oncolytic virus is HSV1716.

13. A kit comprising a predetermined amount of oncolytic virus and a predetermined amount of chemotherapeutic agent, wherein the chemotherapeutic agent is an aurora kinase inhibitor.

14. Products containing therapeutically effective amounts of:
(i) HSV1716, and
(ii) an aurora kinase inhibitor
for simultaneous or sequential use in a method of medical treatment, preferably treatment of cancer.
